(19) United States
(12) Patent Application Publication
(10) Pub. No.: US 2008/0260751 A1
(43) Pub. Date: Oct. 23, 2008

(54) Methods and Compositions for the Modulation of Immune Responses and Autoimmune Diseases

(76) Inventors: Christopher L. Karp, Cincinnati, OH (US); Senad Divanovic, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 10/599,797

(22) PCT Filed: Apr. 14, 2005
(86) PCT No.: PCT/US2005/012931

§ 371 Date: Oct. 18, 2007

Related U.S. Application Data
(60) Provisional application No. 60/562,794, filed on Apr. 16, 2004.
(60) Provisional application No. 60/664,001, filed on Mar. 22, 2005.

(51) Int. Cl. A61K 39/395; A61K 31/7088; A61K 38/17; A61P 37/06; G01N 33/53

(52) U.S. Cl. 424/158.1; 514/44; 514/12; 435/7.8

(57) ABSTRACT

The present invention relates to a regulation of inflammation and immune responses. The present invention relates to a method of treating a condition comprising administering a pharmaceutically effective amount of an activator of RP105. The condition is typically associated with TLR-4 activation and cytokine production. Conditions addressed by the invention include sepsis, septic shock, inflammation, rheumatoid arthritis and Crohn's disease. The invention also provides the use of an activator of RP105 in the manufacture of a medicament for use in the treatment of a condition associated with cytokine production and methods for identifying an activator of RP105, which is also suitable for use in the treatment of a condition associated with stimulus-induced cytokine production. More specifically the patent relates to the use of RP105 as a specific inhibitor of TLR4 signaling and as a physiological regulator of TLR4 signaling for the treatment of TLR4-mediated inflammation and immune-related diseases. This invention also relates to treating a subject having a disease or condition associated with Toll-like receptor 4.

This application claims priority to U.S. Provisional Pat. Appl. Ser. No. 60/562,794, filed Apr. 16, 2004, and U.S. Provisional Pat. Appl. Ser. No. 60/664,001, filed Mar. 22, 2004, both of which are incorporated by reference in their entirety.

### FIELD OF THE INVENTION

The present invention relates to regulation of inflammation and immune responses. The present invention relates to a method of treating a condition comprising administering a pharmaceutically effective amount of a compound that upregulates RP105-mediated inhibition of TLR inhibition, through either upregulating RP105 expression or activity. More specifically the patent relates to the use of RP105 as a specific inhibitor of TLR4 signaling and as a physiological regulator of TLR4 signaling for the treatment of TLR4-mediated inflammation and TLR4-driven immune-processes.

### BACKGROUND OF THE INVENTION

The field of innate immunity has undergone a recent renaissance, fueled largely by the molecular identification of critical receptors and signaling pathways involved in pathogen recognition. Study of the Toll-like receptor (TLR) family has led the way. Activation of TLR signaling by conserved microbial molecular signatures promotes the induction of both innate and adaptive immune responses (1, 2). It has long been clear that such immune responses need to be kept under tight control. Responses that are delayed or of insufficient vigor can lead to a failure to control infection. On the other hand, excessive or inappropriate inflammation can be harmful or even fatal. The hyper-inflammatory responses that characterize sepsis provide a paradigmatic example, as do the more localized inappropriate inflammatory processes leading to inflammatory bowel disease and arthritis (3-7).

The eleven known members of the mammalian TLR family are characterized structurally by an extracellular leucine-rich repeat (LRR) domain, a conserved pattern of juxtamembrane cysteine residues, and an intracytoplasmic signaling domain (Toll/IL-1 resistance [TIR]) that is highly conserved across the TLRs as well as the receptors for IL-1 and IL-18 (2, 8). The TLR-like molecule RP105 was originally cloned as a B cell-specific molecule able to drive B cell proliferation (9, 10). Like TLRs, RP105 has a conserved extracellular LRR domain and a TLR-like pattern of juxtamembrane cysteines (9-13). Unlike the TLRs, however, RP105 lacks a TIR domain, containing a mere 6 to 11 intracytoplasmic amino acids. In parallel with TLR4, whose surface expression and signaling depends upon co-expression of the molecule MD-2, surface expression of RP105 is dependent upon the co-expression of the MD-2 homologue, MD-1 (14-17).

Phylogenetic analysis demonstrates that RP105 is actually a specific homologue of TLR4. Further, RP105 is not B cell-specific as originally proposed: we have found that its expression directly mirrors that of TLR4 on antigen-presenting cells. In Toll and TLR4, mutation of the conserved juxtamembrane cysteine residues, or deletion of the extracellular portion altogether, results in a constitutively active molecule (18-20). This suggests that the activation of Toll/TLRs is normally restrained through extracellular protein/protein interactions, likely through the LRR domain (21). On the other hand, deletions or mutations in the TIR domain of Toll/TLRs can yield inactive or dominant negative molecules (18, 22, 23). Thus, RP105 has the structure of an inhibitory TLR4.

RP105 specifically inhibits TLR4 signaling when co-expressed in cells and RP105 is a physiological regulator of TLR4 signaling and of TLR4-driven proinflammatory responses in vivo. Although the activation of proinflammatory responses through TLRs is critical for both innate and adaptive immunity, excessive or inappropriate inflammation can itself be maladaptive. RP105 inhibits TLR signaling and this regulation of TLR expression provides a point of control (48-50) since RP105 acts specifically to inhibit TLR4 signaling.

The present invention now provides methods of using RP105 as a specific inhibitor of TLR4 signaling to treat TLR4-mediated pathogenic processes and diseases.

### BRIEF SUMMARY OF THE INVENTION

The present invention relates to the modulation of inflammation and immune responses. Methods and compositions for suppressing a pathological inflammatory or immune response are disclosed. The methods involve administering an effective amount of an agent that upregulates RP105-mediated inhibition of TLR inhibition, through either upregulating RP105 expression or activity. The methods are useful in the treatment of diseases marked by excessive or inappropriate inflammatory processes, including autoimmune diseases such as rheumatoid arthritis, juvenile rheumatoid arthritis, spondyloarthropathy, multiple sclerosis and inflammatory bowel disease; pathological systemic responses to a variety of injuries, such as systemic inflammatory response syndrome and adult respiratory distress syndrome; and localized and systemic infectious processes such as sepsis and meningitis.

Specifically, the present invention provides for methods and compositions for upregulating the expression of RP105, or for the engagement of or signaling through RP105, in order to inhibit Toll-like receptor signaling. More specifically, the patent relates to the use of RP105 as a specific inhibitor of TLR4 signaling and as a physiological regulator of TLR4 signaling for the treatment of TLR4-driven inflammatory processes and TRLR4-driven immune-immune pathology. This invention also relates to treating a subject having a disease or condition associated with Toll-like receptor 4 by agonists of RP105.

In another aspect, the invention involves a method for treating a subject having a disease or condition associated with Toll-like receptor 4 comprising administering to the subject a therapeutically or prophylactically effective amount of one or more compounds capable of activating RP105 so that the action and/or expression of Toll-like receptor 4 is inhibited.

Consequently, broadly stated, the present invention provides a method of modulating an immune response comprising modulating the expression or activity of RP105. In one aspect, the present invention provides a method of suppressing an immune response comprising administering an effective amount of an activator of RP105 to a subject in need of such treatment.

In one embodiment, excessive or inappropriate inflammatory or proinflammatory responses through TLRs are treated by modulation of RP105.

In a further embodiment, the present invention provides a method of preventing or treating an autoimmune disease comprising administering an effective amount of an activator of RP105 to a subject having, suspected of having, or susceptible to having an autoimmune disease.

The invention also includes pharmaceutical compositions containing one or more activators of RP105 for use in inducing tolerance in autoimmune, inflammatory or infectious disease

In preferred embodiments of the above methods and compositions, the RP105 activator is an antibody that binds RP105, a small molecule that signals through RP105, or a compound that upregulates RP105 expression. Also in a preferred embodiment of the invention, the activator of RP105 is administered in combination a nucleic acid sequence encoding an RP105 protein.

The invention also includes pharmaceutical compositions containing an RP105 protein or a nucleic acid sequence encoding an RP105 protein for use in suppressing an immune response. Such compositions can include other molecules that can suppress the immune response such as activators of RP105.

The invention further includes screening assays for identifying substances that modulate RP105 expression or activity. Such substances may be useful in the therapeutic methods and compositions of the invention. The invention also includes diagnostic kits and methods for detecting conditions associated with increased, decreased or abnormal expression of RP105.

Further provided are methods for treating a condition involving Toll-like receptor-induced diseases or pathology comprising administering to a patient an effective amount of an agent that activates RP105. Preferably, the Toll-like receptor is Toll-like receptor 4. In another embodiment, the method further comprises administering a therapeutic steroid. In another embodiment, the agent that down-regulates the Toll-like receptor decreases the endogenous amount of intracellular or extracellular cytokine.

The present invention relates to methods for inhibiting Toll-like receptor-4 (“TLR-4”) activity and treatment of Toll-like receptor 4 induced inflammatory bowel disease and related gastrointestinal pathologies. In a further aspect, the present invention concerns the treatment of Toll-like receptor 4 induced inflammatory bowel disease, including ulcerative colitis, Crohn's disease, indeterminate colitis, infectious colitis, drug or chemical-induced colitis, diverticulitis, and ischemic colitis. In another embodiment, the present methods provide for the treatment of Toll-like receptor 4-dependent colitis.

It is one object of the present invention to provide methods for inhibiting the biological activity of TLR-4, as, for example, by inhibiting its expression or signaling. It is a further object of the invention to provide methods of treating those diseases in which inhibiting TLR-4 would have a beneficial effect.

In one embodiment, the Toll-like receptor 4 induced disease is one or more of systemic lupus erythematosis, sceleroderma, Sjogren's syndrome, multiple sclerosis and other demyelinating diseases, rheumatoid arthritis, juvenile rheumatoid arthritis, myocarditis, uveitis, Reiter's syndrome, gout, osteoarthritis, polymyositis, primary biliary cirrhosis, inflammatory bowel disease, Crohn's disease, ulcerative colitis, aplastic anemia, Addison's disease, insulin-dependent diabetes mellitus, and other diseases.

In an alternate embodiment, methods of the present invention are used to inhibit the Toll-like receptor 4 induced disease including atherosclerosis, transplant atherosclerosis, vein-graft atherosclerosis, stent restenosis, and angioplasty restenosis, and to thereby treat the cardiovascular diseases that atherosclerosis causes (hereinafter “vascular diseases”). These methods may be used in any patient who could benefit from reducing atherosclerosis that is already present, from inhibiting atherosclerosis that has yet to form, or from both reducing existing atherosclerosis and inhibiting new atherosclerosis. Such patients include those suffering from, for example, angina pectoris and its subtypes (e.g., unstable angina and variant angina); ischemias affecting organs such as the brain, heart, bone, and intestines, and conditions associated with the ischemias, such as stroke, transient ischemic attacks, heart attack, osteonecrosis, colitis, poor kidney function, and congestive heart failure; poor blood circulation to the extremities and the complications of poor blood circulation, such as slow wound healing, infections, and claudication; atherosclerosis itself, including restenosis following angioplasty or stenting of atherosclerotic lesions; vein-graft atherosclerosis following bypass surgery; transplant atherosclerosis; and other diseases caused by or associated with atherosclerosis.

In another embodiment, such diseases include, for example, vascular disease such as atherosclerosis and thrombosis, restenosis after angioplasty and/or stenting, and vein-graft disease after bypass surgery.

In another aspect, the invention involves a method for treating a condition involving a cytokine-induced diseases or pathology by administering to a patient an effective amount of an agent that down-regulates a Toll-like receptor (TLR). In one embodiment, the agent that down-regulates the Toll-like receptor does so by decreasing the endogenous amount of intracellular or extracellular cytokines.

In one embodiment, the cytokine-mediated disease is selected from acquired immune deficiency syndrome, acute and chronic pain, acute purulent meningitis, adult respiratory distress syndrome (ARDS), Alzheimer's disease, aphthous ulcers, arthritis, asthma, atherosclerosis, atherosclerosisatopic dermatitis, bone resorption diseases, cachexia, chronic obstructive pulmonary disease, congestive heart failure, contact dermatitis, Crohn's disease, dermatoses with acute inflammatory components, diabetes, endotoxemia, glomerulonephritis, graft versus host disease, granulocyte transfusion, Guillain-Barre syndrome, inflammatory bowel disease, leprosy, leukopherisis, malaria, multiple organ injury secondary to trauma, multiple sclerosis, myocardial infarction, necrotizing enterocolitis and syndromes associated with hemodialysis, osteoarthritis, osteoporosis, psoriasis, reperfusion injury, restenosis following percutaneous transluminal coronary angioplasty, rheumatoid arthritis, sarcoidosis, scleroderma, sepsis, septic shock, stroke, systemic lupus erythrematosis, thermal injury, toxic shock syndrome, traumatic arthritis, and ulcerative colitis.

In another embodiment, the present invention provides for methods of treating a disease mediated by cytokines which comprises administering to a patient in need of such treatment a therapeutically effective amount of one or more activators of RP105. In another embodiment, the present invention provides for methods of treating a gastrointestinal disorder in a patient in need thereof comprising administering to the patient a therapeutically effective amount of one or more activators of RP105. In an additional embodiment, the gastrointestinal disorder is an inflammatory bowel disease, Crohn's disease, gastritis, irritable bowel syndrome, ulcerative colitis, a peptic ulcer, a stress ulcer, a bleeding ulcer, gastric hyperacidity, dyspepsia, gastroparesis, Zollinger-Ellison syndrome, gastroesophageal reflux disease, a bacterial infection, short-bowel (anastomosis) syndrome, a hypersecretory state associated with systemic mastocytosis or basophilic leukemia or hyperhistaminemia.

In another aspect, the cytokine involved in the TLR-4-mediated disease or pathology is a proinflammatory cytokine. In still another aspect, the proinflammatory cytokine is selected from the group consisting of TNF-α, IL-1, IL-10, IL-6, and IL-8.

Consequently, broadly stated, the present invention provides a method of modulating an immune response comprising modulating the expression or activity of RP105.

In one aspect, the present invention provides a method of suppressing an immune response comprising administering an effective amount of a modulator of RP105 activation or expression to a subject in need of such treatment.

In one embodiment, the present invention provides a method of inducing immune suppression or tolerance to a transplanted organ or tissue in a recipient subject comprising administering an effective amount of an activator of RP105 activation or expression to the recipient subject prior to the transplantation of the organ or tissue.

In another embodiment, the present invention provides a method of preventing or inhibiting graft versus host disease in a recipient subject receiving an organ or tissue transplant comprising administering an effective amount of an activator of RP105 activation or expression to the organ or tissue prior to the transplantation in the recipient subject.

In yet another embodiment, the present invention provides a method of preventing or inhibiting fetal loss comprising administering an effective amount of an activator of RP105 activation or expression to a subject in need thereof.

In a further embodiment, the present invention provides a method of preventing or treating an autoimmune disease comprising administering an effective amount of an activator of RP105 activation or expression to a subject having, suspected of having, or susceptible to having an autoimmune disease.

In yet a further embodiment, the present invention provides a method of preventing or treating an allergy comprising administering an effective amount of an activator of RP105 activation or expression to a subject having or suspected of having an allergy.

The invention also includes pharmaceutical compositions containing one or more activators of RP105 activation or expression for use in inducing tolerance in transplantation, allergy or autoimmune disease or for preventing or treating fetal loss.

In preferred embodiments of the above methods and compositions, the RP105 activator is an antibody that binds RP105 or an antisense oligonucleotide that activates the expression of RP105. Also in a preferred embodiment of the invention, the inhibitor of RP105 is administered in combination with an MD-1 protein or a nucleic acid sequence encoding an MD-1 protein.

As stated above, activating RP105 can be used to induce immune suppression. Consequently, inhibiting RP105 can be used in preventing immune suppression or inducing an immune response.

Therefore, in another aspect, the present invention provides a method of immune suppression comprising administering an effective amount of an RP105 protein or a nucleic acid sequence encoding an RP105 protein to a subject in need thereof.

The invention also includes pharmaceutical compositions containing an RP105 protein or a nucleic acid sequence encoding an RP105 protein for use in suppressing an immune response. Such compositions can include other molecules that can activate the immune response such as activators of MD-1 activity or expression.

The invention further includes screening assays for identifying substances that modulate RP105 expression or activity. Such substances may be useful in the therapeutic methods and compositions of the invention. The invention also includes diagnostic kits and methods for detecting conditions associated with increased, decreased or abnormal expression of RP105.

In one embodiment, the present invention provides for a method for screening a test compound for the potential to prevent, stabilize, or treat an autoimmune or inflammatory disease comprising the steps of: a) contacting a first cell sample from a first subject that has, or is at risk for developing, an autoimmune or inflammatory disease with the test compound; b) contacting a second cell sample from a second subject that does not have, or is not predisposed to developing, an autoimmune or inflammatory disease with the test compound, wherein the first and second subjects are of the same species and the first and second cell samples are contacted with the test compound in the same manner; and c) measuring RP105 inhibition of TLR4 in the first and second samples, wherein the compound is determined to have the potential if TLR4 inhibition in the first sample is decreased relative to the second sample. In one embodiment, RP105 inhibition of TLR4 is measured by the ability of the lipopolysaccharide (LPS) signaling complex to bind LPS. In another embodiment, RP105 activation or expression is measured.

In another embodiment, the present invention provides for a method for screening a test compound for the potential to prevent, stabilize, or treat an autoimmune or inflammatory disease comprising the steps of: a) contacting a population of cells from a subject that has, or is at risk for developing, an autoimmune or inflammatory disease with the test compound; b) contacting a second cell element from the subject with the test compound, wherein the cells and the second cell element are contacted with the test compound in the same manner; and c) measuring RP105 activation or expression of the cells, wherein the test compound is determined to have the potential if the RP105 activation or expression of the cell increases relative to the second cell element. In another embodiment, the cells are autoimmune cells. In another embodiment, the cells are leukocytes.

In another embodiment, the present invention provides for a method for diagnosing an autoimmune or inflammatory disease, or a predisposition to the disease, in a subject comprising the steps of: a) obtaining a first cell sample from a first subject; b) obtaining a second cell sample from a second subject of the same species as the first subject, wherein the second subject does not have, or is not at risk for developing, the autoimmune disease; c) contacting the first cell sample and the second cell sample with a compound that preferentially increases the activation or expression of RP105, wherein both of the first and second samples are contacted with the compound in the same manner; and d) measuring the activation or expression of RP105 in the first cell sample and in the second cell sample, wherein a decrease in activation or expression of RP105 in the first sample relative to the activation or expression of RP105 in the second sample indicates that the first subject has, or is predisposed to developing, the autoimmune disease.

In another embodiment, the present invention provides for a method for diagnosing an autoimmune disease, or a predisposition to the disease, in a subject comprising the steps of: a) contacting autoimmune cells from a subject that has an autoimmune disease, or is at risk for developing an autoimmune disease, with the test compound; b) contacting a second cell element from the subject with the test compound, wherein the autoimmune cells and the second cell element are contacted with the compound in the same manner; and c) measuring the activation of RP105 of the autoimmune cells, wherein the subject has, or is at risk for developing, the autoimmune disease if the activation or expression of RP105 of the autoimmune cells decreases relative to the second cell element.

In another embodiment, the present invention provides for a method for the stratification of a human patient into a therapeutic subgroup for an autoimmune disease comprising the steps of: a) contacting a cell sample from the patient with a compound that preferentially decreases the activation of RP105 of leukocytes; b) measuring the activation of RP105 of the leukocytes; and c) placing the patient into a therapeutic subgroup based on the amount of decrease of the activation or expression of RP105.

In another embodiment, the present invention provides for a method for monitoring a therapy for a human that has an autoimmune disease, or is at risk for developing the disease, comprising the steps of: i) obtaining a first cell sample from the patient and contacting the first cell sample with a compound that preferentially decreases the activation of RP105 of leukocytes; ii) measuring the activation of RP105 of leukocytes in the first cell sample; iii) obtaining a second cell sample from the patient and contacting the second cell sample with the compound, wherein the second cell sample is obtained at least 12 hours after obtaining the first cell sample; iv) measuring the activation of RP105 of leukocytes in the second cell sample; and v) determining the efficacy of the therapy based on leukocyte RP105 activation or expression, wherein an increase in the activation or expression indicates that the therapy is efficacious.

In another embodiment, the present invention provides for a pharmaceutical composition for use in suppressing an immune response comprising an activator of RP105/MD-1 complex formation in admixture with a suitable diluent or carrier. In one embodiment, the activator is an antibody that binds MD-1, RP105, or both.

In another embodiment, the present invention provides for a screening method for identifying an immunostimulatory compound, comprising: contacting a functional RP105 with a test compound under conditions which, in absence of the test compound, permit a negative control response mediated by a RP105 signal transduction pathway; detecting a test response mediated by the RP105 signal transduction pathway; and determining the test compound is an immunostimulatory compound when the test response exceeds the negative control response.

In another embodiment, the present invention provides for a screening method for identifying an immunostimulatory compound, comprising: contacting a functional RP105 with a test compound under conditions which, in presence of a reference immunostimulatory compound, permit a reference response mediated by a RP105 signal transduction pathway; detecting a test response mediated by the RP105 signal transduction pathway; and determining the test compound is an immunostimulatory compound when the test response equals or exceeds the reference response.

In another embodiment, the present invention provides for a screening method for identifying a compound that modulates RP105 signaling activity, comprising: contacting a functional RP105 with a test compound and a reference immunostimulatory compound under conditions which, in presence of the reference immunostimulatory compound alone, permit a reference response mediated by a RP105 signal transduction pathway; detecting a test-reference response mediated by the RP105 signal transduction pathway; determining the test compound is an agonist of RP105 signaling activity when the test-reference response exceeds the reference response; and determining the test compound is an antagonist of RP105 signaling activity when the reference response exceeds the test-reference response.

In another embodiment, the present invention provides for a screening method for identifying species specificity of an immunostimulatory compound, comprising: measuring a first species-specific response mediated by a RP105 signal transduction pathway when a functional RP105 of a first species is contacted with a test compound; measuring a second species-specific response mediated by the RP105 signal transduction pathway when a functional RP105 of a second species is contacted with the test compound; and comparing the first species-specific response with the second species-specific response.

In one embodiment, the screening method is performed on a plurality of test compounds. In another embodiment, the response mediated by the RP105 signal transduction pathway is measured quantitatively. In another embodiment, the functional RP105 is expressed in a cell. In another embodiment, the cell is an isolated mammalian cell that naturally expresses the functional RP105.

In another embodiment, the cell is an isolated mammalian cell that does not naturally express the functional RP105, and wherein the cell comprises an expression vector for RP105. In another embodiment, the cell is a human fibroblast. In another embodiment, the cell comprises an expression vector comprising an isolated nucleic acid which encodes a reporter construct selected from the group of interleukins, NF-kappaB, interferons, and TNFs. In another embodiment, the functional RP105 is part of a cell-free system. In another embodiment, the reference immunostimulatory compound is a nucleic acid. In another embodiment, the nucleic acid is a CpG nucleic acid. In another embodiment, the reference immunostimulatory compound is a small molecule. In another embodiment, the test compound is a part of a combinatorial library of compounds. In another embodiment, the test compound is a nucleic acid. In another embodiment, the nucleic acid is a CpG nucleic acid. In another embodiment, the test compound is a small molecule. In another embodiment, the test compound is a polypeptide. In another embodiment, the response mediated by a RP105 signal transduction pathway is induction of a reporter gene under control of a promoter response element selected from the group consisting of ISRE, IL-6, IL-8, IL-12 p40, IFN-alpha, IFN-beta, IFN-omega, TNF, IP-10, and I-TAC. In another embodiment, the response mediated by a RP105 signal transduction pathway is selected from the group consisting of (a) induction of a reporter gene under control of a minimal promoter responsive to a transcription factor selected from the group consisting of AP1, NF-kappaB, ATF2, IRF3, and IRF7; (b) secretion of a chemokine; and (c) secretion of a cytokine. In another embodiment, the response mediated by a RP105 signal transduction pathway is secretion of a type 1 IFN. In another embodiment, the response mediated by a RP105 signal transduction pathway is secretion of a chemokine. In another embodiment, the contacting a functional RP105 with a test compound further comprises, for each test compound, contacting with the test compound at each of a plurality of concentrations. In another embodiment, the e detecting is performed 6-12 hours following the contacting. In another embodiment, the detecting is performed 16-24 hours following the contacting.

The present compounds may also be used in co-therapies, partially or completely, in place of other conventional anti-inflammatories, such as together with steroids, cyclooxygenase-2 inhibitors, NSAIDs, DMARDS, antibiotics, immunosuppressive agents, 5-lipoxygenase inhibitors, LTB₄ antagonists and LTA4 hydrolase inhibitors and anti-cell adhesion molecules such as anti E-selectin.

In one embodiment, this aspect additionally involves administering a therapeutic steroid to the patient. By way of non-limiting example, therapeutic steroids may include, for example, corticoids, glucocorticoids, dexamethasone, prednisone, prednisalone, and betamethasone.

These methods may employ the compounds of this invention in a monotherapy or in combination with an anti-inflammatory or immunosuppressive agent. Such combination therapies include administration of the agents in a single dosage form or in multiple dosage forms administered at the same time or at different times.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

Throughout this document, all temperatures are given in degrees Celsius, and all percentages are weight percentages unless otherwise stated. All publications mentioned herein are incorporated herein by reference for the purpose of describing and disclosing the compositions and methodologies, which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

### BRIEF DESCRIPTION OF THE FIGURES

FIG. 1 RP105 expression by human peripheral blood leukocytes. Flow cytometric analysis of PBMC from healthy human donors. (a) B cells; (b) monocytes; (c) myeloid DC; (d) plasmacytoid DC. Myeloid DC were identified as lineage negative (CD3⁻, CD14⁻, CD19⁻, CD20⁻, CD56⁻), HLA-DR⁺, CD11c⁺. Plasmacytoid DC were identified as lineage negative, HLA-DR⁺, CD11c⁻, BDCA-4⁺. Data are representative of an experimental n>15 for monocytes; n=3 for the other cell types.

FIG. 2 RP105 expression by murine leukocytes. Flow cytometric analysis of cell populations. (a) splenic B cells (representative of an experimental n>50). (b) resident peritoneal macrophages (n=5). (c) splenic CD11c⁺CD11b⁺CD4⁻ and CD11c⁺CD11b⁺CD4⁺DC (n=6). (d) splenic CD11c⁺CD11b⁻CD8α⁺DC (n=6). (e) bone marrow-derived DC (n=6). (f) splenic plasmacytoid DC (CD19⁺B220+CD11c⁺GR-1⁺) [n=5]; red: isotype control, green: TLR4, blue: RP105. Analysis of splenic DC subsets was performed after 10 d of in vivo treatment with flt3L.

FIG. 3 Dose-dependent suppression of TLR4 signaling in HEK293 cells by RP105 expression. (a) HEK293 cells stably expressing CD14 were transiently transfected with cDNA encoding MD-1, MD-2, TLR4, empty vector control cDNA (EV) and/or RP105. Cells were subsequently stimulated with purified E. coli K235 LPS (10 ng/ml). (b) HEK293 cells stably expressing CD14 and TLR4 were transiently transfected with MD-1 and MD-2 along with the indicated concentrations of RP105 and/or EV cDNA, and secondarily stimulated with LPS (10 ng/ml). (c) HEK293 cells stably expressing MD-2 and TLR4 were transiently co-transfected with an NF-κB-firefly luciferase reporter plasmid, a TK-renilla luciferase reporter plasmid and MD-1, along with EV (open bars) or RP105 (filled bars). Cells were stimulated with the indicated concentrations of LPS. *p<0.03, **p<0.004, compared with RP105-deficient cells. Means +/−SE of triplicate cultures in a single experiment are depicted, representative of an experimental n=4 (a and b); n=2 (c). NS: no stimulation.

FIG. 4 Specificity of RP105-mediated suppression. (a) HEK293 cells stably expressing CD14 and TLR4 (open bars) or CD14, TLR4 and RP105 (filled bars) were transiently transfected with MD-1 and MD-2, and subsequently stimulated with purified E. coli K235 LPS (10 ng/ml) or IL-1β (100 ng/ml). (b) HEK293 cells stably expressing CD14 and TLR2 were transiently transfected with MD-1 and EV (open bars) or MD-1 and RP105 (filled bars) and subsequently stimulated with Zymosan A (10 μg/ml) or IL-1β (100 ng/ml). *p<0.0001, **p=0.05, compared with RP105-deficient cells. Means +/−SE of triplicate cultures in a single experiment are depicted, representative of an experimental n=2-4.

FIG. 5 The extracellular domain of RP105 is sufficient to effect suppression of TLR4 signaling. (a) HEK293 cells stably expressing CD14 and TLR4 were transiently transfected with MD-2 and MD-1, along with EV, RP105 or the extracellular domain of RP105 (EC-RP105), as indicated. Cells were subsequently stimulated with purified E. coli K235 LPS (10 ng/ml). Means +/−SE of triplicate cultures in a single experiment are depicted, representative of an experimental n=4 *p<0.02, **p<0.002, compared with RP105-deficient cells. (b, c) HEK293 cells stably expressing CD14 and TLR2 were transiently transfected with MD-2 and MD-1, along with EV or EC-RP105, as indicated. Cells were subsequently stimulated with (b) Zymosan A (10 μg/ml) or (c) IL-1 (100 ng/ml) as noted. Means +/−SE of replicate cultures (n=9) are depicted. NS, not significant.

FIG. 6 RP105-MD-1 interacts directly with TLR4-MD-2. HEK93 cells stably expressing CD14 (lane 1) or CD14 and FLAG-tagged TLR4 (lanes 2-4) were transiently transfected with MD-2, RP105 and/or MD-1 constructs, as indicated. Lysates were immunoprecipitated with antibodies to FLAG or HA, and the association between RP105-MD-1 and TLR4-MD-2 was examined by immunoblotting using antibodies to FLAG, HA and MD-1, as indicated. The expression of RP105, MD-1, TLR4, and MD-2 in cell lysates was characterized by immunoblotting using anti-FLAG, anti-HA and anti-MD-1 antibodies, as indicated. Data are from a single experiment, representative of an n=3.

FIG. 7 RP105-MD-1 inhibits LPS binding to TLR4-MD-2. HEK293 cells stably expressing CD14 and FLAG-tagged TLR4 (a), or HEK293FT cells (b), were transiently transfected with empty vector (EV), MD-2, RP105, MD-1 and/or TLR4 constructs, as indicated. After incubation with biotinylated LPS, cells were lysed, lysates were immunoprecipitated with streptavidin-conjugated Sepharose A beads, and the association of LPS with (a) TLR4-MD-2 or (b) RP105-MD-1 was examined by immunoblotting using anti-FLAG or anti-HA antibodies, respectively. The expression of RP105, MD-1, TLR4, and MD-2 in cell lysates was characterized by immunoblotting using anti-FLAG and anti-HA antibodies as indicated. Data are from a single experiment, representative of an n=2 (a), or 1 (b).

FIG. 8 Altered TLR4-induced cytokine production by dendritic cells from RP105-deficient mice. Bone marrow-derived DC from wild-type (open symbols) or RP105-deficient (filled symbols) mice were stimulated with purified E. coli K235 LPS (a-d) or CpG DNA (e). Supernatants were harvested after 24 h. (a) TNF. (b) IL-12p70. (c) IL-6. (d) IP-10. (e) TNF. *p<0.05, **p<0.001, ***p<0.01, ****p<0.0001. Means +SE of triplicate cultures in a single experiment, representative of an n=8 (a); 4 (b, c); 7 (d); or 3 (e).

FIG. 9 Ability of heterologous TLR signaling to overcome RP105-mediated inhibition of TLR4 signaling. Bone marrow-derived DC from wild-type (open symbols) or RP105-deficient (filled symbols) mice were stimulated with: (a) commercial E. coli K235 LPS; or (b) purified E. coli K35 LPS plus the TLR2 agonist, Pam₃Cys. *p<0.05, **p<0.001. Means +SE of triplicate cultures in a single experiment; representative of an experimental n=4.

FIG. 10 Exaggerated in vivo responses to LPS in RP105^{-/-} mice. (a) Wild-type mice (n=17) (open bars) or RP105-deficient mice (n=18) (filled bars) were challenged intraperitoneally with 25 μg of purified E. coli K235 LPS. Serum was harvested 60 min later. (b) Wild type (open symbols) or RP105^{-/-} mice (filled symbols) were challenged with 8 mg/kg of purified E. coli K235 LPS (n=7/group). Data represent means +SE. *p<0.0005, **p<0.00005, ***p<10⁻⁸, ****p<0.01.

FIG. 11 MD-1 expression is necessary for RP105-mediated suppression of TLR4 signaling. (a) HEK293 cells stably expressing CD14 and TLR4 were transiently transfected with MD-2; along with EV, MD-1 and/or RP105 as indicated. Cells were subsequently stimulated with purified E. coli K235 LPS (10 ng/ml). Means +/−SE of triplicate cultures in a single experiment are depicted, representative of an experimental n=4. *p<0.008. (b) HEK293 cells were analyzed for surface and intracellular RP105 expression by FACS (representative of 2 separate experiments).

FIG. 12 RP105 fails to inhibit NF-κB transactivation driven by overexpression of TLR4 signaling molecules. HEK293FT cells were co-transfected with an NF-κB-firefly luciferase reporter plasmid (pELAM; 0.5 μg), along with plasmids encoding an I-κB super-repressor, RP105 (100 ng) plus MD-1 (50 ng), Mal (100 ng), IRAK-1 (100 ng), Myd88 (100 ng), TRIF (100 ng) and/or empty vector (EV; 100 ng). After 48 h, cells were lysed and luciferase activity was quantified. Means +/−SE of triplicate cultures in a single experiment are depicted, representative of an experimental n=3.

FIG. 13 MD-1 and MD-2 can interact directly with each other. HEK293T cells were transiently transfected with MD-2 and/or MD-1 constructs, as indicated. Lysates were immunoprecipated with antibodies to FLAG or HA, and the association between MD-1 and MD-2 was examined by immunoblotting using the antibodies indicated.

FIG. 14 Similar expression of molecules regulating TLR signaling in bone marrow-derived dendritic cells from RP105-deficient and wild-type mice. mRNA expression was analyzed by quantitative RT-PCR in bone marrow-derived DC from wild type (open symbols) or RP105^{-/-} (filled symbols) mice prior to and following stimulation with 10 ng of purified E. coli K235 LPS. (a) IRAK-M, (b) Tollip, (e) ST2, (d) SIGIRR.

FIG. 15 Altered TLR4-induced cytokine production by resident peritoneal macrophages from RP105-deficient mice. Resident peritoneal macrophages from wild type (open symbols) or RP105-deficient (filled symbols) mice were stimulated with purified E. coli K235 LPS. Supernatants were harvested after 24 h. *p<0.005, **p<0.05.

FIG. 16 Regulation of RP105 expression by LPS. DC were stimulated with purified E. coli K235 LPS. Kinetic analysis of RP105 and TLR4 mRNA expression was performed by quantitative RT-PCR. (a) Regulation of TLR4 and RP105 expression in mouse bone marrow-derived DC. Means +SE of duplicate PCR reactions in a single experiment, representative of 3 different experiments. (b) Regulation of TLR4 and RP105 expression in human monocyte-derived DC. Means +SE of duplicate PCR reactions from 3 different donors. RP105 expression: square symbols; TLR4 expression: round symbols. RP105-deficient mice: filled symbols.

FIG. 17 Genotyping and phenotyping of RP105-deficient mice. (a) Genotyping via standard techniques. (b) FACS analysis of peripheral blood cells.

### DETAILED DESCRIPTION OF THE INVENTION

Before the present device and methods for tissue augmentation is described, it is to be understood that this invention is not limited to the specific methodology, devices, formulations, and compositions described as such may, of course, vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “and”, and “the” include plural referents unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

As used herein, the following terms shall have the definitions given below.

The term “activation” as used herein to describe the mechanism of action of an activator of RP105, means a mechanism wherein a compound upregulates RP105-mediated inhibition of TLR activity, through upregulating RP105 expression, increasing engagement of or signaling activity with TLR and/or other means that provide for a decrease in the activity of TLR.

The term “administration” of the pharmaceutically active compounds and the pharmaceutical compositions defined herein includes systemic use, as by injection (especially parenterally), intravenous infusion, suppositories and oral administration thereof, as well as topical application of the compounds and compositions. Oral administration is particularly preferred in the present invention.

“Ameliorate” or “amelioration” means a lessening of the detrimental effect or severity of the cell adhesion disorder in the subject receiving therapy, the severity of the response being determined by means that are well known in the art.

“Chemokines” are chemotactic cytokines that are released by a wide variety of cells to attract macrophages, T-cells, eosinophils, basophils, neutrophils and endothelial cells to sites of inflammation and tumor growth. There are two main classes of chemokines, the CXC-chemokines and the CC-chemokines. The class depends on whether the first two cysteines are separated by a single amino acid (CXC-chemokines) or are adjacent (CC-chemokines). The CXC-chemokines include interleukin-8 (IL-8), neutrophil-activating protein-1 (NAP-1), neutrophil-activating protein-2 (NAP-2), GROα, GROβ, GROγ, ENA-78, GCP-2, IP-10, MIG and PF4. CC chemokines include RANTES, MIP-1α, MIP-2β, monocyte chemotactic protein-1 (MCP-1), MCP-2, MCP-3 and eotaxin.

By “compatible” herein is meant that the components of the compositions which comprise the present invention are capable of being commingled without interacting in a manner which would substantially decrease the efficacy of the pharmaceutically active compound under ordinary use conditions.

By “corticosteroid” is meant any naturally occurring or synthetic steroid hormone that can be derived from cholesterol and is characterized by a hydrogenated cyclopentanoperhydrophenanthrene ring system. Naturally occurring corticosteriods are generally produced by the adrenal cortex. Synthetic corticosteriods may be halogenated. Functional groups required for activity include a double bond at Δ4, a C3 ketone, and a C20 ketone. Corticosteroids may have glucocorticoid and/or mineralocorticoid activity.

Exemplary corticosteroids include, for example, dexamethasone, betamethasone, triamcinolone, triamcinolone acetonide, triamcinolone diacetate, triamcinolone hexacetonide, beclomethasone, dipropionate, beclomethasone dipropionate monohydrate, flumethasone pivalate, diflorasone diacetate, fluocinolone acetonide, fluorometholone, fluorometholone acetate, clobetasol propionate, desoximethasone, fluoxymesterone, fluprednisolone, hydrocortisone, hydrocortisone acetate, hydrocortisone butyrate, hydrocortisone sodium phosphate, hydrocortisone sodium succinate, hydrocortisone cypionate, hydrocortisone probutate, hydrocortisone valerate, cortisone acetate, paramethasone acetate, methylprednisolone, methylprednisolone acetate, methylprednisolone sodium succinate, prednisolone, prednisolone acetate, prednisolone sodium phosphate, prednisolone tebutate, clocortolone pivalate, flucinolone, dexamethasone 21-acetate, betamethasone 17-valerate, isoflupredone, 9-fluorocortisone, 6-hydroxydexamethasone, dichlorisone, meclorisone, flupredidene, doxibetasol, halopredone, halometasone, clobetasone, diflucortolone, isoflupredone acetate, fluorohydroxyandrostenedione, beclomethasone, flumethasone, diflorasone, fluocinolone, clobetasol, cortisone, paramethasone, clocortolone, prednisolone 21-hemisuccinate free acid, prednisolone metasulphobenzoate, prednisolone terbutate, and triamcinolone acetonide 21-palmitate. By “low dose corticosteroid” is meant a dose that is less than a dose that would typically be given to a patient for treatment of inflammation. Exemplary low doses of corticosteroids are as follows: cortisol: 12 mg/day; cortisone: 15 mg/day; prednisone: 3 mg/day; methylprednisolone: 2.5 mg/day; triameinolone: 2.5 mg/day; betamethasone: 250 μg/day; dexamethasone: 450 μg/day; hydrocortisone: 9 mg/day.

The term “patient” or “subject,” as used herein, is intended to encompass any mammal, animal or human subject, which may benefit from treatment with the compounds, compositions and methods of the present invention.

“Pharmaceutically-acceptable” shall mean that the pharmaceutically active compound and other ingredients used in the pharmaceutical compositions and methods defined herein are suitable for use in contact with the tissues of humans and lower animals without undue toxicity, irritation, allergic response, and the like, commensurate with a reasonable benefit/risk ratio.

The term “prodrug” indicates a therapeutic agent that is prepared in an inactive form that is converted to an active form (i.e., drug) within the body or cells thereof by the action of endogenous enzymes or other chemicals and/or conditions.

The phrase “safe and effective amount” means a sufficient amount of pharmaceutically active compound to effect the activation of RP105 and inhibition of TLR-4. Within the scope of sound medical judgment, the required dosage of a pharmaceutically active agent or of the pharmaceutical composition containing that active agent will vary with the severity of the condition being treated, the duration of the treatment, the nature of adjunct treatment, the age and physical condition of the patient, the specific active compound employed, and like considerations discussed more fully hereinafter. In arriving at the “safe and effective amount” for a particular compound, these risks must be taken into consideration, as well as the fact that the compounds described herein provide pharmaceutical activity at lower dosage levels than conventional compounds.

“Toll-like receptors” or “TLRs” are type I transmembrane proteins containing repeated leucine-rich motifs in their extracellular domains. At least eight mammalian TLR proteins have been identified, Toll-like receptors 1-8. Ligand engagement of the TLRs results in activation of NF-κB and induction of the cytokines and co-stimulatory molecules required for the activation of the adaptive immune response. Human Toll-like receptor 4 (also known as TLR4 and hToll), the human homolog to the Drosophilae protein known as Toll, was cloned from a human fetal liver/spleen library, characterized, and mapped to chromosome 9q32-33. Toll-like receptor 4 mRNA expression can be detected in the cells of the immune system: monocytes, macrophages, dendritic cells, γΔT-cells, Th1 and Th2αβT-cells, and B-cells. Expression has also been detected in the cardiac myocytes and placenta.

“Treat,” “treating,” “treatment,” and “therapy” as used herein refer to any curative therapy, prophylactic therapy, ameliorative therapy and preventative therapy of a subject. “Ameliorate” or “amelioration” means a lessening of the detrimental effect or severity of a disease or pathology in the subject receiving therapy, the severity of the response being determined by means that are well known in the art.

As used herein, unless otherwise indicated, the terms “bacterial infection(s)” include bacterial infections that occur in mammals, fish and birds as well as disorders related to bacterial infections that may be treated or prevented by administering antibiotics such as the compounds of the present invention. Such bacterial infections, and disorders related to such infections, include the following: pneumonia, otitis media, sinusitus, bronchitis, tonsillitis, and mastoiditis related to infection by Streptococcus pneumonia, Haemophilus influenzae, Moraxella catarrhalis, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and Toxic shock syndrome), or Groups A, B, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenzae, or Listeria spp.; disseminated Mycobacterium avium complex (MAC) disease related to infection by Mycobacterium avium, or Mycobacterium intracellulare; gastroenteritis related to infection by Campylobacter jejuni; odontogenic infection related to infection by viridans streptococci; persistent cough related to infection by Bordetella pertussis; gas gangrene related to infection by Clostridium perfringensor Bacteroides spp.; and atherosclerosis related to infection by Helicobacter pylori or Chlamydia pneumoniae. Bacterial infections and protozoa infections and disorders related to such infections that may be treated or prevented in animals include the following: bovine respiratory disease related to infection by P. haem., P. multocida, Mycoplasma bovis, or Bordetella spp.; cow enteric disease related to infection by E. coli (i.e., coccidia, cryptosporidia, etc.); dairy cow mastitis related to infection by Staph. aureus, Strep. uberis, Strep. agalactiae, Strep. dysgalactiae, Klebsiella spp., Corynebacterium, or Enterococcus spp.; swine respiratory disease related to infection by A. pleuro., P. multocida, or Mycoplasma spp.; swine enteric disease related to infection by E coli, Lawsonia intracellularis, Salmonella, or Serpulina hyodyisinteriae; cow footrot related to infection by Fusobacterium spp.; cow metritis related to infection by E coli; cow hairy warts related to infection by Fusobacterium necrophorum or Bacteroides nodosus; cow pink-eye related to infection by Moraxella bovis; urinary tract infection in dogs and cats related to infection by E coli; skin and soft tissue infections in dogs and cats related to infection by Staph. epidermidis, Staph. intermedius, coagulase neg. Staph. or P. multocida; and dental or mouth infections in dogs and cats related to infection by Alcaligenes spp., Bacteroides spp., Clostridium spp., Enterobacter spp., Eubacterium, Peptostreptococcus, Porphyromonas, or Prevotella. Other bacterial infections and disorders related to such infections that may be treated or prevented in accord with the method of the present invention.

Accordingly the invention provides a method of treating a condition associated with cytokine production in a mammal comprising administering a pharmaceutically effective amount of an activator of a member of RP105. The invention also provides the use of an activator of RP105 in the manufacture of a medicament for use in the treatment of a condition associated with cytokine production

In one embodiment the cytokine is TNF, preferably TNFα. In another embodiment the cytokine is IL-1, preferably IL-1β. The cytokine-associated condition may be any one of sepsis, septic shock, inflammation, rheumatoid arthritis or Crohn's disease. The cytokine-associated condition may be irritable bowel disease (IBD). The cytokine-associated condition may be ulcerative colitis.

In a preferred embodiment the cytokine-associated condition is induced by a Toll-related receptor (TRR) ligand. In another preferred embodiment the cytokine-associated condition is induced by lipopolysaccharide (LPS). Gram-negative bacteria may induce the condition. Gram-positive bacteria may induce the condition. In another preferred embodiment the cytokine-associated condition is induced by zymosan. The condition may induced by yeast. The TRR ligand may be a microbial factor.

The invention also provides a pharmaceutical formulation comprising a pharmaceutically acceptable carrier and an activator of RP105 identifiable by a method of the invention.

The invention also provides the use of an activator of RP105 to study the inhibition of sepsis, septic shock and/or inflammation caused by a TRR-ligand, preferably LPS, in vitro.

The invention provides a method of treating sepsis or septic shock in a subject administering a pharmaceutically effective amount of an activator of RP105.

The invention provides a method of treating inflammation in a subject comprising administering a pharmaceutically effective amount of an activator of RP105. The invention provides a RP105 activator for use as a medicament. The invention provides a RP105 activator for use in the manufacture of a medicament to treat sepsis, septic shock and/or inflammation. The invention provides a method or activator of the invention wherein the activator is a vaccine, an antibody or a fragment thereof which is capable of binding RP105 or a fragment thereof.

The invention provides a method of treating sepsis, septic shock and/or inflammation in a subject comprising administering a pharmaceutically effective amount of an activator of a member of RP105. The invention provides a method of treating rheumatoid arthritis in a subject comprising administering a pharmaceutically effective amount of an activator of a member of RP105. The invention provides a method of treating Crohn's disease in a subject comprising administering a pharmaceutically effective amount of an activator of a member of RP105. The invention provides a method of treating a condition induced by a TRR ligand in a subject comprising administering a pharmaceutically effective amount of an activator of a member of RP105.

The invention provides a method of treating a condition induced by a TLR4 ligand in a subject comprising administering a pharmaceutically effective amount of an activator of a member of RP105. The invention provides a method of treating a condition induced by a pathogen in a subject comprising administering a pharmaceutically effective amount of an activator of a member of RP105. The invention provides a method of treating a condition induced by bacteria, or a factor derived there from in a subject comprising administering a pharmaceutically effective amount of an activator of a member of RP105.

All of the results of the inventors demonstrate that RP105 is an immune modulating molecule that has utility in a wide range of applications. Accordingly, the present invention includes all uses that relate to the realization of the immune modulatory properties of RP105 including, but not limited to, the development of therapeutic and diagnostic assays and compositions as well as the preparation and/or isolation of other molecules that modulate RP105 that may be useful in the therapeutic and diagnostic assays and compositions of the invention.

Inducing Immune Suppression

In one aspect, the present invention provides a method of suppressing an immune response comprising administering an effective amount of an activator of RP105 to a subject in need of such treatment. The invention includes a use of an effective amount of an activator of RP105 to suppress an immune response or to prepare a medicament to suppress an immune response.

The term “an activator of RP105” means any molecule or compound that can activate the expression of the RP105 gene or that can activate the activity of the RP105 protein.

In another embodiment, the activator of RP105 is an RP105 specific antibody. Antibodies to RP105 may be prepared using techniques known in the art such as those described by Kohler and Milstein, Nature 256, 495 (1975) and in U.S. Pat. Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kenneft, McKearn, and Bechtol (eds.), 1980, and Antibodies: A Laboratory Manual, Harlow and Lane (eds.), Cold Spring Harbor Laboratory Press, 1988, which are also incorporated herein by reference). Within the context of the present invention, antibodies are understood to include monoclonal antibodies, polyclonal antibodies, antibody fragments (e.g., Fab, and F(ab′).sub.2) and recombinantly produced binding partners.

The therapeutic methods of the invention can be used to treat any condition wherein it is desirable to modulate RP105 expression or activity. Such conditions include, but are not limited to, autoimmune disease, inflammatory conditions, septic shock, organ dysfunction, neurodegenerative diseases (e.g., Alzheimer's disease), stroke and spinal injury.

As stated previously, the method of the present invention may also be used to treat or prevent autoimmune disease. In an autoimmune disease, the immune system of the host fails to recognize a particular antigen as “self” and an immune reaction is mounted against the host's tissues expressing the antigen. Normally, the immune system is tolerant to its own host's tissues and autoimmunity can be thought of as a breakdown in the immune tolerance system.

Accordingly, in a further embodiment, the present invention provides a method of preventing or treating an autoimmune disease comprising administering an effective amount of an activator of RP105 to a subject having, suspected of having, or susceptible to having an autoimmune disease. The invention includes a use of an effective amount of an activator of RP105 to prevent or activate an autoimmune disease or to prepare a medicament to prevent or activate an autoimmune disease.

Autoimmune diseases that may be treated or prevented according to the present invention include, but are not limited to, type 1 insulin-dependent diabetes mellitus, adult respiratory distress syndrome, inflammatory bowel disease, dermatitis, meningitis, thrombotic thrombocytopenic purpura, Sjogren's syndrome, encephalitis, uveitic, leukocyte adhesion deficiency, rheumatoid arthritis, rheumatic fever, Reiter's syndrome, psoriatic arthritis, progressive systemic sclerosis, primary biniary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, antigen-antibody complex mediated diseases, autoimmune haemolytic anemia, Hashimoto's thyroiditis, Graves disease, habitual spontaneous abortions, Reynard's syndrome, glomerulonephritis, dermatomyositis, chronic active hepatitis, celiac disease, autoimmune complications of AIDS, atrophic gastritis, ankylosing spondylitis and Addison's disease.

The above described methods for suppressing an immune response using RP105 activators may be further enhanced by co-administering other immune modulators including but not limited to RP105, anti-fgl2, anti-B7, anti-CD80 or anti-CD86. Preferably, the RP105 activators are co-administered with an RP105 protein or a nucleic acid molecule encoding an RP105 protein as described in the PCT application no. WO00/12130 and U.S. Pat. No. 5,780,609, which are incorporated herein by reference in their entirety.

Preventing Immune Suppression

The term “RP105 protein” includes the full-length RP105 protein as well as fragments or portions of the protein. Preferred fragments or portions of the protein are those that are sufficient to induce an immune response or prevent immune suppression. The RP105 protein or the nucleic acid encoding the RP105 protein can be readily obtained by one of skill in the art. The RP105 protein or nucleic acid may be modified from the known sequences to make it more useful in the methods of the present invention.

In one embodiment, the RP105 protein is prepared as a soluble fusion protein. The fusion protein may contain the extracellular domain of RP105 linked to an immunoglobulin (Ig) Fc Region. The RP105 fusion may be prepared using techniques known in the art. Generally, a DNA sequence encoding the extracellular domain of RP105 is linked to a DNA sequence encoding the Fc of the Ig and expressed in an appropriate expression system where the RP105-Fclg fusion protein is produced. The RP105 protein may be obtained from known sources or prepared using recombinant DNA techniques. The protein may have any of the known published sequences for RP105. For example, the sequences can be obtained from GenBank as described above. The protein may also be modified to contain amino acid substitutions, insertions and/or deletions that do not alter the immunomodulatory properties of the protein. Conserved amino acid substitutions involve replacing one or more amino acids of the RP105 amino acid sequence with amino acids of similar charge, size, and/or hydrophobicity characteristics. When only conserved substitutions are made the resulting analog should be functionally equivalent to the RP105 protein. Non-conserved substitutions involve replacing one or more amino acids of the RP105 amino acid sequence with one or more amino acids that possess dissimilar charge, size, and/or hydrophobicity characteristics.

Administration of an “effective amount” of the RP105 protein and nucleic acid of the present invention is defined as an amount effective, at dosages and for periods of time necessary to achieve the desired result. The effective amount of the RP105 protein or nucleic acid of the invention may vary according to factors such as the disease state, age, sex, and weight of the animal. Dosage regime may be adjusted to provide the optimum therapeutic response. For example, several divided doses may be administered daily or the dose may be proportionally reduced as indicated by the exigencies of the therapeutic situation.

In one embodiment, the present invention provides a method of inducing fetal loss comprising administering an effective amount of an RP105 protein or a nucleic acid sequence encoding an RP105 protein to a subject in need thereof. RP105 Modulators

The present invention also includes the isolation and/or identification of substances to modulate RP105 expression or activity. Such substances or RP105 modulators may be useful in the above-described therapeutic methods. Two examples of RP105 modulators include antibodies and antisense molecules that are described in detail above. Other RP105 modulators may be identified, for example, using the screening assays described below.

Substances that Bind RP105

Substances that affect RP105 activity can be identified based on their ability to bind to RP105.

Substances that can bind with the RP105 of the invention may be identified by reacting the RP105 with a substance that potentially binds to RP105, and assaying for complexes, for free substance, or for non-complexed RP105, or for activation of RP105.

Accordingly, the invention provides a method of identifying substances that can bind with RP105, comprising the steps of: (a) reacting RP105 and a test substance, under conditions that allow for formation of a complex between the RP105 and the test substance, and (b) assaying for complexes of RP105 and the test substance, for free substance or for non complexed RP105, wherein the presence of complexes indicates that the test substance is capable of binding RP105.

Conditions that permit the formation of substance and RP105 complexes may be selected having regard to factors such as the nature and amounts of the substance and the protein.

The substance-protein complex, free substance or non-complexed proteins may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against RP105 or the substance, or labeled RP105, or a labeled substance may be utilized. The antibodies, proteins, or substances may be labeled with a detectable substance as described above.

RP105, or the substance used in the method of the invention may be insolubilized. For example, RP105 or substance may be bound to a suitable carrier. Examples of suitable carriers are agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling. The proteins or substance may also be expressed on the surface of a cell in the above assay. The invention also contemplates assaying for an antagonist or agonist of the action of RP105.

It will be understood that the agonists and antagonists that can be assayed using the methods of the invention may act on one or more of the binding sites on the protein or substance including agonist binding sites, competitive antagonist binding sites, non-competitive antagonist binding sites or allosteric sites.

The invention also makes it possible to screen for antagonists that activate the effects of an agonist of RP105. Thus, the invention may be used to assay for a substance that competes for the same binding site of RP105.

Peptide Mimetics

The present invention also includes peptide mimetics of the RP105 of the invention. For example, a peptide derived from a binding domain of RP105 will interact directly or indirectly with an associated molecule in such a way as to mimic the native binding domain. Such peptides may include competitive activators, enhancers, peptide mimetics, and the like. All of these peptides as well as molecules substantially homologous, complementary or otherwise functionally or structurally equivalent to these peptides may be used for purposes of the present invention.

“Peptide mimetics” are structures that serve as substitutes for peptides in interactions between molecules (See Morgan et al. (1989), Ann. Reports Med. Chem. 24:243-252 for a review). Peptide mimetics include synthetic structures that may or may not contain amino acids and/or peptide bonds but retain the structural and functional features of a peptide, or enhancer or activator of the invention. Peptide mimetics also include peptoids, oligopeptoids (Simon et al. (1972) Proc. Natl. Acad, Sci USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of activator peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides of the invention may also be used to identify lead compounds for drug development. The structure of the peptides described herein can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess.

Drug Screening Methods

In accordance with one embodiment, the invention enables a method for screening candidate compounds for their ability to increase or decrease the activity of a RP105 protein. The method comprises providing an assay system for assaying RP105 activity, assaying the activity in the presence or absence of the candidate or test compound and determining whether the compound has increased or decreased RP105 activity.

Accordingly, the present invention provides a method for identifying a compound that affects RP105 protein activity or expression comprising: (a) incubating a test compound with a RP105 protein or a nucleic acid encoding a RP105 protein; and (b) determining an amount of RP105 protein activity or expression and comparing with a control (i.e. in the absence of the test substance), wherein a change in the RP105 protein activity or expression as compared to the control indicates that the test compound has an effect on RP105 protein activity or expression.

In accordance with a further embodiment, the invention enables a method for screening candidate compounds for their ability to increase or decrease expression of a RP105 protein. The method comprises putting a cell with a candidate compound, wherein the cell includes a regulatory region of a RP105 gene operably joined to a reporter gene coding region, and detecting a change in expression of the reporter gene.

In one embodiment, the present invention enables culture systems in which cell lines which express the RP105 gene, and thus RP105 protein products, are incubated with candidate compounds to test their effects on RP105 expression. Such culture systems can be used to identify compounds that upregulate or down-regulate RP105 expression or its function, through the interaction with other proteins.

Such compounds can be selected from protein compounds, chemicals and various drugs that are added to the culture medium. After a period of incubation in the presence of a selected test compound(s), the expression of RP105 can be examined by quantifying the levels of RP105 mRNA using standard Northern blotting procedure to determine any changes in expression as a result of the test compound. Cell lines transfected with constructs expressing RP105 can also be used to test the function of compounds developed to modify the protein expression. In addition, transformed cell lines expressing a normal RP105 protein could be mutagenized by the use of mutagenizing agents to produce an altered phenotype in which the role of mutated RP105 can be studied in order to study structure/function relationships of the protein products and their physiological effects.

Accordingly, the present invention provides a method for identifying a compound that affects the binding of an RP105 protein and an RP105 binding protein comprising: (a) incubating (i) a test compound; (ii) an RP105 protein and (iii) an RP105 binding protein under conditions which permit the binding of RP105 protein to the RP105 binding protein; and (b) assaying for complexes of RP105 protein and the RP105 binding protein and comparing to a control (i.e. in the absence of the test substance), wherein a reduction of complexes indicates that the compound has an effect on the binding of the RP105 protein to an RP105 binding protein.

All testing for novel drug development is well suited to defined cell culture systems, which can be manipulated to express RP105 and study the result of RP105 protein modulation. Animal models are also important for testing novel drugs and thus may also be used to identify any potentially useful compound affecting RP105 expression and activity and thus physiological function.

Compositions

The invention also includes pharmaceutical compositions containing substances that activate RP105 activators for use in immune suppression as well as pharmaceutical compositions containing substances that enhance RP105 for use in preventing immune suppression. Substances that activate RP105 include substances that activate RP105 gene expression as well as substances that activate RP105 activity. Such substances include antisense molecules to RP105, antibodies to RP105 as well as other substances or RP105 antagonists identified using the screening assays described herein.

Substances that enhance RP105 include substances that enhance RP105 expression and/or activity. Such substances include nucleic acid molecules encoding RP105, RP105 proteins and other substances or RP105 agonists identified using the screening assays described herein.

Such pharmaceutical compositions can be for intralesional, intravenous, topical, rectal, parenteral, local, inhalant or subcutaneous, intradermal, intramuscular, intrathecal, transperiton al, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to patients, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids. The pharmaceutical compositions may additionally contain other agents such as immunosuppressive drugs or antibodies to enhance immune tolerance or immunostimulatory agents to enhance the immune response.

In one embodiment, the pharmaceutical composition for use in inducing immune tolerance comprises an effective amount of an activator of RP105 in admixture with a pharmaceutically acceptable diluent or carrier. The RP105 activator is preferably an antisense oligonucleotide to RP105 or an antibody that binds to RP105. The pharmaceutical compositions may also contain other active agents such as other immune modulators including, but not limited to RP105, fgl2, B7, CD80 or CD86 including antagonists, agonists and modulators thereof. Preferably the compositions further contain an RP105 protein or a nucleic acid molecule encoding an RP105 protein.

Pharmaceutical compositions comprising nucleic acid molecules may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as co-precipitation and incorporation of DNA into liposomes. Recombinant molecules may also be delivered in the form of an aerosol or by lavage. The nucleic acid molecules of the invention may also be applied extracellularly such as by direct injection into cells.

In another aspect, the pharmaceutical composition for use in preventing immune suppression comprises an effective amount of an RP105 protein or a nucleic acid encoding an RP105 protein in admixture with a pharmaceutically acceptable diluent or carrier. Such compositions may be administered either alone or in combination with other active agents such as RP105 activators.

Diagnostic Assays

The finding by the present inventors that RP105 is involved in immune regulation allows the detection of conditions involving an increase or decrease in RP105 activity or expression resulting in an aberrant or inappropriate immune response. Such conditions include, but are not limited to, habitual fetal loss, autoimmune diseases, allergies, immune deficiency diseases, graft rejection, inflammatory conditions, wound healing, neurodegenerative diseases, stroke, spinal injury and conditions that lead to septic shock and organ dysfunction in critically ill patients.

Accordingly, the present invention provides a method of detecting a condition associated with increased or decreased RP105 expression or activity (including an absence) comprising assaying a sample for (a) a nucleic acid molecule encoding a RP105 protein or a fragment thereof or (b) an RP105 protein or a fragment thereof and comparing the amount of RP105 nucleic acid or protein detected with a suitable control.

Nucleic Acid Molecules

Nucleotide probes can be prepared based on the sequence of RP105 for use in the detection of nucleotide sequences encoding RP105 or fragments thereof in samples, preferably biological samples such as cells, tissues and bodily fluids. The probes can be useful in detecting the presence of a condition associated with RP105 or monitoring the progress of such a condition. Accordingly, the present invention provides a method for detecting a nucleic acid molecules encoding RP105 comprising contacting the sample with a nucleotide probe capable of hybridizing with the nucleic acid molecule to form a hybridization product, under conditions which permit the formation of the hybridization product, and assaying for the hybridization product.

A nucleotide probe may be labeled with a detectable substance such as a radioactive label, which provides for an adequate signal and has sufficient half-life such as 32P, 3H, 14C or the like. Other detectable substances that may be used include antigens that are recognized by a specific labeled antibody, fluorescent compounds, enzymes, antibodies specific for a labeled antigen, and chemiluminescence. An appropriate label may be selected having regard to the rate of hybridization and binding of the probe to the nucleic acid to be detected and the amount of nucleic acid available for hybridization. Labeled probes may be hybridized to nucleic acids on solid supports such as nitrocellulose filters or nylon membranes as generally described in Sambrook et al., 1989, Molecular Cloning, A Laboratory Manual (2nd ed.). The nucleotide probes may be used to detect genes, preferably in human cells, that hybridize to the nucleic acid molecule of the present invention preferably, nucleic acid molecules which hybridize to the nucleic acid molecule encoding RP105 under stringent hybridization conditions as described herein.

Nucleic acid molecules encoding a RP105 protein can be selectively amplified in a sample using the polymerase chain reaction (PCR) methods and cDNA or genomic DNA. It is possible to design synthetic oligonucleotide primers from the nucleotide sequence of RP105 for use in PCR. A nucleic acid can be amplified from cDNA or genomic DNA using oligonucleotide primers and standard PCR amplification techniques. The amplified nucleic acid can be cloned into an appropriate vector and characterized by DNA sequence analysis. cDNA may be prepared from mRNA, by isolating total cellular mRNA by a variety of techniques, for example, by using the guanidinium-thiocyanate extraction procedure of Chirgwin et al., Biochemistry, 18, 5294-5299 (1979). cDNA is then synthesized from the mRNA using reverse transcriptase (for example, Moloney MLV reverse transcriptase available from Gibco/BRL, Bethesda, Md., or AMV reverse transcriptase available from Seikagaku America, Inc., St. Petersburg, Fla.).

Proteins

The RP105 protein may be detected in a sample using antibodies that bind to the protein as described in detail above. Accordingly, the present invention provides a method for detecting a RP105 protein comprising contacting the sample with an antibody that binds to RP105 which is capable of being detected after it becomes bound to the RP105 in the sample.

Antibodies specifically reactive with RP105, or derivatives thereof, such as enzyme conjugates or labeled derivatives, may be used to detect RP105 in various biological materials, for example they may be used in any known immunoassays which rely on the binding interaction between an antigenic determinant of RP105, and the antibodies. Examples of such assays are radioimmunoassays, enzyme immunoassays (e.g. ELISA), immunofluorescence, immunoprecipitation, latex agglutination, hemagglutination and histochemical tests. Thus, the antibodies may be used to detect and quantify RP105 in a sample in order to determine its role in particular cellular events or pathological states, and to diagnose and treat such pathological states.

In particular, the antibodies of the invention may be used in immuno-histochemical analyses, for example, at the cellular and sub-subcellular level, to detect RP105, to localize it to particular cells and tissues and to specific subcellular locations, and to quantitate the level of expression.

Cytochemical techniques known in the art for localizing antigens using light and electron microscopy may be used to detect RP105. Generally, an antibody of the invention may be labeled with a detectable substance and RP105 may be localized in tissue based upon the presence of the detectable substance. Examples of detectable substances include various enzymes, fluorescent materials, luminescent materials and radioactive materials. Examples of suitable enzymes include horseradish peroxidase, biotin, alkaline phosphatase, b-galactosidase, or acetylcholinesterase; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include radioactive iodine I-125, I-131 or 3-H. Antibodies may also be coupled to electron dense substances, such as ferritin or colloidal gold, which are readily visualized by electron microscopy.

Indirect methods may also be employed in which the primary antigen-antibody reaction is amplified by the introduction of a second antibody, having specificity for the antibody reactive against RP105. By way of example, if the antibody having specificity against RP105 is a rabbit IgG antibody, the second antibody may be goat anti-rabbit gamma-globulin labeled with a detectable substance as described herein.

Where a radioactive label is used as a detectable substance, RP105 may be localized by autoradiography. The results of autoradiography may be quantitated by determining the density of particles in the autoradiographs by various optical methods, or by counting the grains.

The preferred embodiments are exemplified by the following examples.

### EXAMPLES

Activation of Toll-like receptor (TLR) signaling by microbial signatures is critical to the induction of immune responses. Such responses demand tight regulation. RP105 is a TLR homolog, thought to be largely B cell-specific, which lacks a signaling domain. The present invention demonstrates that RP105 expression is wide, directly mirroring that of TLR4 on antigen presenting cells. Furthermore, RP105 is a specific inhibitor of TLR4 signaling in HEK293 cells, a function conferred by its extracellular domain. Notably, RP105 and its helper molecule, MD-1, interact directly with the TLR4 signaling complex, inhibiting the ability of this complex to bind microbial ligand. Finally, we demonstrate that RP105 is a physiological regulator of TLR4 signaling in dendritic cells, and of endotoxicity and Leishmania major infection in vivo.

Activation of TLR signaling by conserved microbial molecular signatures promotes the induction of both innate and adaptive immune responses. It has long been clear that such immune responses need to be kept under tight control. Responses that are delayed or of insufficient vigor can lead to a failure to control infection. On the other hand, excessive or inappropriate inflammation can be harmful or even fatal. The hyper-inflammatory responses that characterize sepsis provide a paradigmatic example, as do the more localized inappropriate inflammatory processes leading to inflammatory bowel disease and arthritis.

Mammalian TLRs are characterized structurally by an extracellular leucine-rich repeat (LRR) domain, a conserved pattern of juxtamembrane cysteine residues, and an intracytoplasmic signaling domain (Toll/IL-1 resistance [TIR]) that is highly conserved across the TLRs as well as the receptors for IL-1 and IL-18. The TLR-like molecule RP105 was originally cloned as a B cell-specific molecule able to drive B cell proliferation. Like TLRs, RP105 has a conserved extracellular LRR domain and a TLR-like pattern of juxtamembrane cysteines. Unlike the TLRs, however, RP105 lacks a TIR domain, containing a mere 6-11 intracytoplasmic amino acids (depending upon the prediction algorithm). In parallel with TLR4, whose surface expression and signaling depends upon co-expression of the secreted extracellular protein MD-2, surface expression of RP105 is dependent upon the co-expression of the MD-2 homolog, MD-1.

Here, we show that RP105 is a specific homolog of TLR4. We further show that RP105 is not B cell-specific as originally proposed: RP105 protein expression directly mirrors that of TLR4 on antigen-presenting cells. In Toll and TLR4, mutation of the conserved juxtamembrane cysteine residues, or significant deletion of the extracellular portion, has been shown to result in a constitutively active molecule. This suggests that Toll/TLR activation is normally restrained through extracellular protein/protein interactions, likely through the LRR domain. On the other hand, deletions or mutations in the TIR domain of Toll/TLRs can yield inactive or dominant negative molecules. Thus, RP105 has the apparent structure of an inhibitory TLR4 and RP105 is a physiological regulator of TLR4 signaling. RP105 is a specific inhibitor of TLR4 signaling, a function conferred by its extracellular domain. Without wishing to be bound by theory in any way, it is believed that the RP105/MD-1 complex interacts directly with TLR4/MD-2, inhibiting the ability of this LPS signaling complex to bind LPS. RP105 is a physiological regulator of TLR4 signaling in primary dendritic cells, and of responses to endotoxin as well as Leishmania infection in vivo.

RP105, a TLR4 homolog whose expression mirrors that of TLR4 on antigen presenting cells, is a negative regulator of TLR4 signaling. RP105 specifically inhibits TLR4 signaling when co-expressed in HEK293 cells. Further, RP105 is a physiological regulator of endotoxin-driven TLR4 signaling, as well as of endotoxicity in vivo. Finally, RP105-mediated counter-regulation is relevant in the context of infection with a well-described model (and human) pathogen: RP105 modulates the course of L. major infection, restraining both innate and adaptive immune responses. Although the activation of proinflammatory responses through TLRs is critical for host defense, excessive or inappropriate inflammation can itself be maladaptive. RP105 joins a growing list of molecules and processes that have been shown to inhibit TLR signaling. RP105 stands out for its apparent specificity for inhibition of TLR4 signaling. This specificity, together with the structural homologies between RP105 and TLR4 provides a mechanism of negative regulation of TLR4 signaling by RP105: interference with TLR4 signaling through direct interactions of RP105/MD-1 with the TLR4/MD-2 cell surface signaling complex. Co-immunoprecipitation experiments revealed direct physical association between RP105/MD-1 and TRL4/MD-2, an association that inhibits LPS binding to this signaling complex. The exact stoichiometry of RP105/MD-1/MD-2/TLR4 interactions remains to be defined. We show that RP105 regulates LPS responses differently in myeloid cells and B cells. As noted, treatment with antibodies to RP105 does not activate human monocyte/macrophages, failing, for example, to drive proinflammatory cytokine production. As for LPS-driven B cell responses, while LPS-induced murine B cell proliferation is strictly dependent upon TLR4, B cells from RP105 knockout mice have reduced LPS-driven proliferative responses as well as diminished humoral immune responses when LPS is used as an adjuvant for vaccination with T cell-dependent antigens. It is reasonable to suspect that the dichotomous effects of RP105 on TLR4 signaling in B cells and myeloid cells are due to differential interactions with cell surface molecular partners in these different cell types. Widely differing levels of expression of TLR4 (significant on myeloid cells; hardly detectable on B cells) may provide the key. TLR4 multimerization appears to be necessary for signaling. While TLR4/MD-2 would be expected to have a higher affinity for homodimerization than for heterodimerization with RP105/MD-1, data presented here suggest the likelihood that both homo- and heterodimers can multimerize with further TLR4/MD-2 complexes. This suggests a model whereby: (a) when TLR4/MD-2 is highly expressed (e.g., on myeloid cells), lower affinity heterodimeric interactions inhibit TLR4 multimerization and signaling; but (b) when TLR4 is limiting (e.g., on B cells), such heterodimeric interactions serve to facilitate further TLR4 recruitment and signaling. The fact that RP105 appears to promote B cell activation, while inhibiting DC activation, suggests the possibility that, overall, immunoregulation by RP105 leads to augmentation of humoral immunity (through effects on B cells) along with concomitant inhibition of cellular immune responses (through effects on DCs and macrophages).

The apparent TLR4-specificity of RP105 raises another issue. Concurrent stimulation of TLR4 and other TLRs is able to effect reversal RP105-mediated inhibition of TLR4 signaling. Clearly, most microbes that express ligands that stimulate TLR4 signaling also express ligands that stimulate signaling through other TLRs. TLR4 is notable for leading to a more robust and complex response, both in terms of signaling and in terms of subsequent gene expression, than the other TLRs. TLR4 may also stand out among the TLRs for its ability to signal in response to a variety of endogenous “danger signals”. The ability of TLR4 to recognize endogenous heat shock proteins and extracellular matrix components unmasked by tissue injury suggests the possibility RP105-mediated modulation of the in vivo response to L. major infection may not be a function of the expression of yet-to-be discovered leishmanial ligands for TLR4, but of the inflammation-induced expression of host-derived TLR ligands. More generally, such considerations raise the possibility that RP105 may well be of special importance in down-modulation of the injurious inflammatory responses observed in the systemic inflammatory response syndrome and in autoimmune diseases. In this regard, it is notable that RP105^{-/-} mice spontaneously develop splenomegaly with age (data not shown).

Materials and Methods

### Methods

Reagents. Zymosan A and commercial LPS were from Sigma. Highly purified E. coli LPS was generated as described⁴⁴. Pam₃Cys was from EMC Microcollections. CpG (5′ TCCATGACGTTCCTGATGCT 3′) was from TriLink Biotechnologies. Recombinant cytokines were from Peprotech. All reagents contacting cultured cells were endotoxin-free to the limits of detection of the Limulus amebocyte lysate assay (Bio-Whittaker) at the concentrations employed, unless otherwise stated.

Cloning and Expression Constructs. Human RP105 and MD-1 were cloned from primary human monocytes by RT-PCR. RP105 deletion mutants and epitope-tagged RP105 and MD-1 constructs, were generated by PCR. The HSV thymidine kinase promoter-driven Renilla luciferase reporter plasmid (pRL-TK) was from Promega; the NF-κB-dependent ELAM-1 promoter-driven Firefly Luciferase plasmid (P-ELAM) has been described²³. cDNA for TLR4 was from R. Medzhitov (Yale Univ.); that for MD-2 was from K. Miyake (Univ. of Tokyo). Plasmids encoding Mal, MyD88, IRAK-1 and TRIF were from K. Fitzgerald (Univ. of Massachusetts). The IκB super-repressor expression plasmid was from R. Hay (Univ. of St. Andrews).

Cell lines. HEK293 cell lines stably expressing CD14, CD14-TLR4, CD14-TLR2, and TLR4-MD-2 have been described^{23,45}. HEK293 cells stably expressing RP105-CD14-TLR4 were generated from CD14-TLR4 HEK293 cells. Transient transfections were performed using PolyFect (Qiagen). Construct expression was quantified by flow cytometry. All cell lines were Mycoplasma-free.

In vitro stimulation. 24 h after transient transfection, HEK293 cells were washed and stimulated for an additional 24 h. Cell-free supernatants were collected and assayed for IL-8 production (ELISA; Pharmingen). To assay NF-κB-driven luciferase expression²³, cells were co-transfected with pELAM (0.5 μg) and pRL-TK (0.1 μg) plasmids, stimulated for 5 h, lysed, and luciferase activity was quantified on a Monolight 3010 luminometer (Pharmingen).

Immunoprecipitation and Western Blotting. 48 h after transfection, HEK293 cells were washed and lysed. For immunoprecipitation (IP) with HA antibody (Y-11; Santa Cruz), cell lysates were incubated with antibody, followed by incubation with protein-G Sepharose beads (Zymed). For IP with FLAG mAb, cell lysates were incubated with anti-FLAG (M2) affinity gel (Sigma). Immunoprecipitates and lysates were resolved by SDS-PAGE, and electrotransferred onto Immobilon-P PVDF membranes (Millipore). After blocking, proteins were revealed by probing with unconjugated M2 mAb (FLAG; Sigma); Y-11 rabbit polycolonal Ab (HA; Santa Cruz); Ab-1 rabbit polyclonal Ab (MD-1; Oncogene); followed by horseradish peroxidase-conjugated secondary mAb (Santa Cruz), LPS labeling, and IP with biotinylated LPS, was performed as described²⁹.

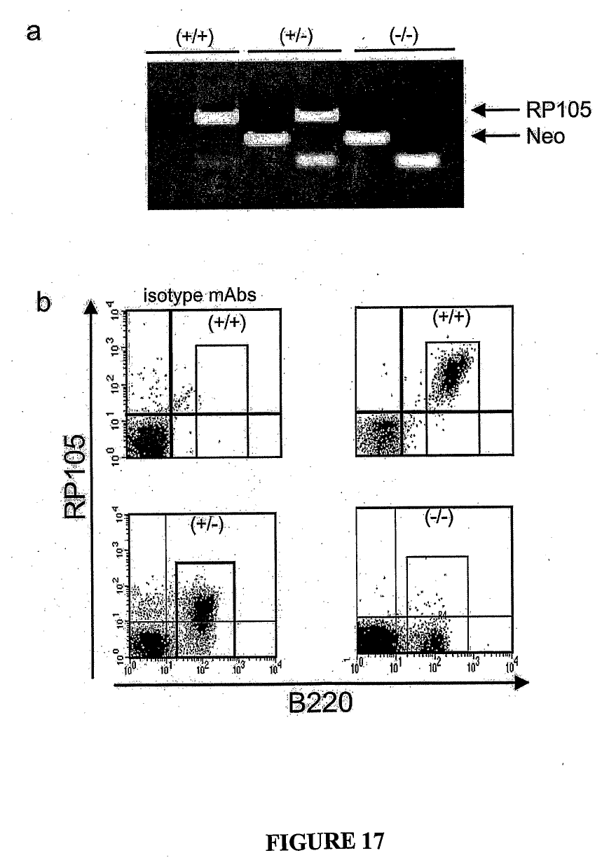

Mice. RP105-deficient mice, on a C57BL/6 background (>10 generations) have been described²⁵. Mice were genotyped by PCR, as well as phenotyped for RP105 surface expression on peripheral blood B cells 25 (FIG. 17). In vivo DC amplification was performed through daily i.p. administration of 10 μg of flt3 ligand (provided by Immunex/Amgen) for 10 d. Mice were housed in a specific pathogen-free facility in high-efficiency particulate-filtered laminar flow hoods with free access to food and water. Animal care was provided in accordance with National Institutes of Health guidelines. These studies were approved by the CCHMC IACUC.

Ex vivo stimulation. Bone marrow-derived DC (BMDDC), generated using standard protocols³¹, were stimulated for 24 h. Cell-free supernatants were collected and assayed by ELISA for TNF (Becton Dickinson), IL-12p70, IL-6 and IP-10 (R&D Systems). RP105, TLR4, IRAK-M, SIGIRR, Tollip and ST2 mRNA expression was analyzed by quantitative RT-PCR in BMDDC prior to and following stimulation with 10 ng of purified E. coli K235 LPS. PCR (LightCycler; Roche) used the following primers: (1) RP105: AGTCTCCTCCCCATCTTGTCC, GATAGCGTCACATCGGAGAGC; (2) TLR4: CATCCAGGAAGGCTTCCACA, GGCGATACAATTCCACCTGC; (3) IRAK-M: GAGAATTGCTCTGGTCCTGGG, CACCTCAAGTGGGAAGCTGG; (4) SIGIRR: GGCCCCTAATTTCCTTTCCC, CATGGAGGCTGAAGTGGCTT; (5) Tollip: TGGACCCACATCACCATCC, GTTGGCATCAGGACCACAGG; (6) ST2: GGCTCTCACTTCTTGGCTGATG, GCCAGACAGTCATATTCCAGGG; (7) β-actin: GGCCCAGAGCAAGAGAGGTA, GGTTGGCCTTAGGGTTCAGG.

In vivo stimulation. Six-8 week old mice were challenged i.p. with 25 μg of purified E. coli K235 LPS. One h later, serum was collected. TNF concentrations were assayed by ELISA. For endotoxicity studies, mice were challenged i.p. with 8 mg/kg of purified E. coli K235 LPS. Clinical endotoxicity was scored using a quantitative scale integrating 4 cardinal signs of systemic toxicity⁴⁶ (piloerection, ocular discharge, lethargy, diarrhea; each scored from 0-3) by an investigator blinded to mouse genotype.

Human leukocytes. PBMC were isolated from healthy volunteers by Ficoll/Hypaque sedimentation. Monocytes were purified by countercurrent elutriation from PBMC isolated by leukapheresis⁴⁷. Purified monocytes were differentiated into DC using standard techniques using IL-4 and GM-CSF. RP105 and TLR4 mRNA expression was analyzed by qRT-PCR in human monocyte-derived DC using the following primers: (1) RP105: TCAGTGCTGCCAATTTCCC, CTGCAGCAGTCAGAAGCCTCT; (2) TLR4: AGTTTCCTGCAATGGATCAAGG, GGACCGACACACCAATGATG; (3) Ubiquitin: CACTTGGTCCTGCGCTTGA, CAATTGGGAATGCAACAACTTTAT. These studies were approved by the CCHMC and University of Cincinnati College of Medicine IRBs.

Flow Cytometry. Surface and intracellular protein expression by HEK293 cells was quantified by FACS techniques described previously⁴⁸. Flow cytometric analysis was performed using a FACSCalibur along with CellQuest Software (Becton Dickinson). At least 20,000 events were acquired for each data point.

Phylogenetic and domain analysis. Sequence alignments were performed using the ClustalW⁴⁹ and Pileup⁵⁰ programs. MEMSAT, SOSUI and SABLE software were used to predict transmembrane boundaries.

Statistical analysis. Data analysis was performed using the unpaired Student t test, or ANOVA with post-hoc analysis by Student t test or Fisher's PLSD, as appropriate.

### Results.

RP105 Expression Mirrors that of TLR4

Phylogenetic analysis of the TLR family has revealed 5 TLR subfamilies: TLR4; TLR3; TLR5; TLR7, TLR8, TLR9; and TLR2, TLR1, TLR6, TLR10². Similar analytic techniques unequivocally place RP105 in the TLR4 subfamily. The domain structure of the 641 amino acid mature RP105 protein has previously been reported^{7,9,10,13}. The extracellular portion of this type I transmembrane protein contains 22 leucine-rich repeats (repeats 7-10 being atypical), along with a pattern of juxtamembrane cysteines that is conserved among the Toll and TLR families. Prediction algorithms disagree on the C-terminal boundary of the transmembrane domain. Although the 6-11 amino acid intracellular domain of RP105 contains 1-2 tyrosine residues, it is devoid of conserved motifs that would suggest potential sites for phosphorylation.

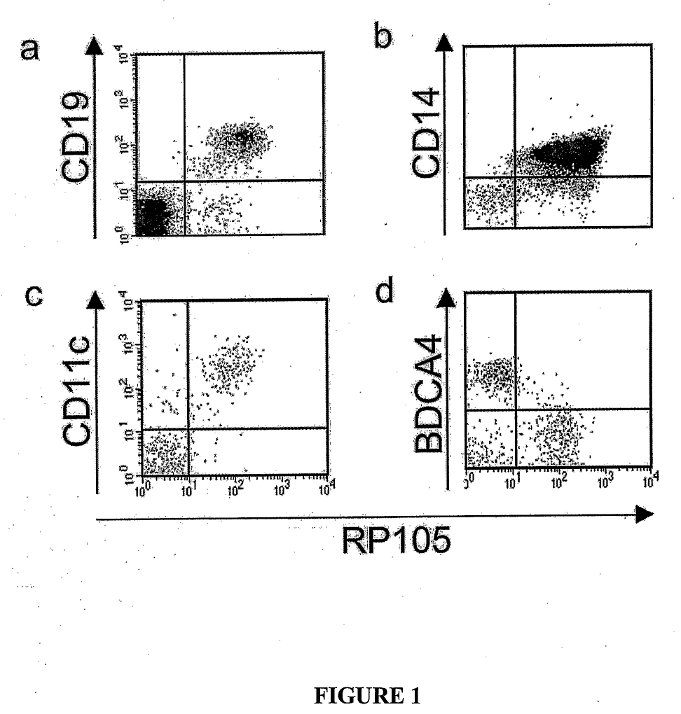

RP105 is reported as a B cell-specific molecule in mice⁷. Further, despite the fact that RP105 is expressed at the mRNA level in primary human myeloid cells⁸⁻¹⁰, the only published examination of RP105 protein expression in such cells (by immune blot analysis) was reported to be negative¹⁰. We examined RP105 expression by FACS in human and murine monocytic cells. In neither species was RP105 expression limited to B cells. In humans, RP105 was also expressed by human monocytes, as well as myeloid dendritic cells (DC) (FIG. 1a-c), cells that express considerably more TLR4 than do B cells²⁰ (and data not shown). RP105 was not expressed by human plasmacytoid DC (FIG. 1d), cells that also fail to express TLR4²⁰⁻²² (and data not shown).

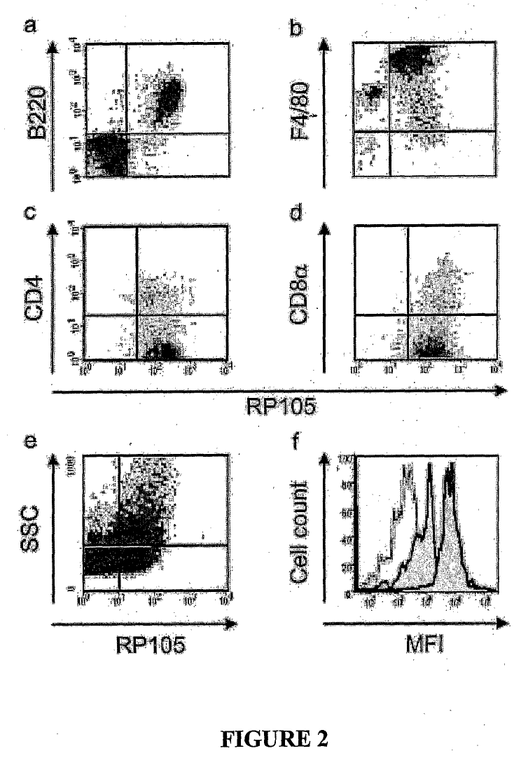

Similarly, FACS analysis revealed that mice express RP105 on resident peritoneal macrophages, splenic DC subsets, as well as bone marrow-derived DC (FIG. 2). To confirm the specificity of the mAb used in this work, RP105-deficient mice were examined alongside wild-type mice in these studies; no RP105 staining was found in B cells, macrophages or DCs from RP105 knockout mice (data not shown). Further, to rule out any potential artifacts (from, e.g., a soluble isoform of RP105; yet to be described), RT-PCR analysis of bone marrow-derived DC was performed to confirm endogenous RP105 expression by such cells (data not shown). Unlike those found in human peripheral blood, essentially all of the splenic plasmacytoid DC from flt3L-treated mice expressed both RP105 and TLR4 (FIG. 2e; and data not shown). Thus, RP105 is not B cell-specific, and its expression directly mirrors that of TLR4 on human and murine macrophages and DC.

RP105 Suppresses TLR4 Signaling in HEK293 Cells

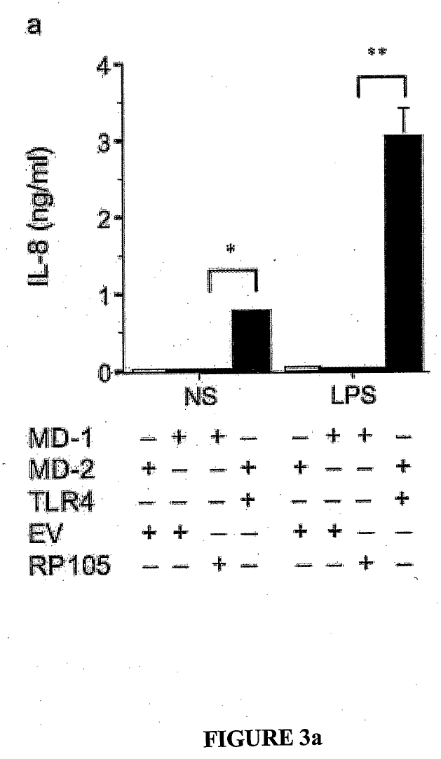

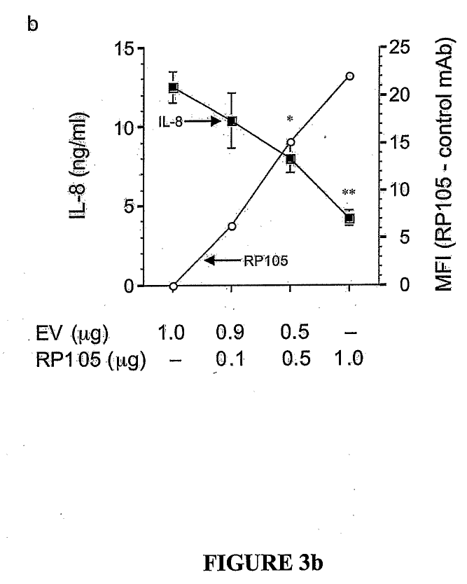

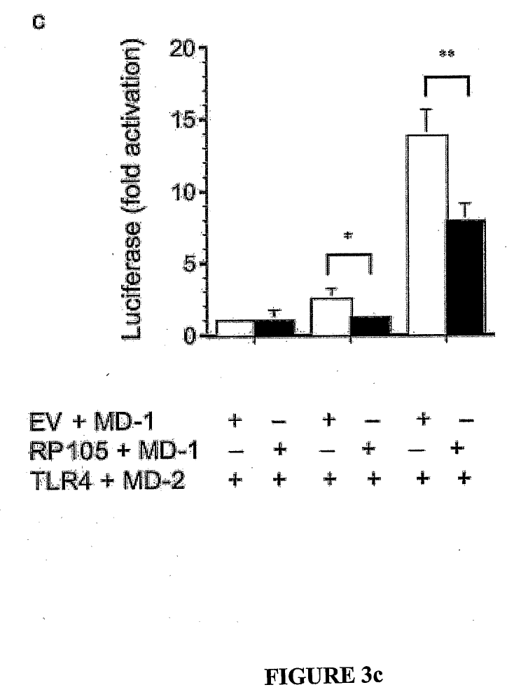

HEK293 cells lack expression of endogenous TLR2, TLR4, TLR9, MD-2 and CD14²³, as well as RP105 and MD-1 (data not shown). Their TLR signaling machinery is fully functional, however²³. As a result, HEK293 cells are used extensively for the in vitro analysis of TLR function^{23,24}. Given the homology of RP105 to TLR4, we first examined whether RP105-MD-1 could act as a signaling receptor for LPS in HEK293 cells. HEK293 cells that stably express CD14 were transiently transfected with cDNA encoding MD-1, MD-2, RP105, and/or TLR4. Although TLR4-MD-2 expression conferred LPS-sensitivity with resultant LPS-driven IL-8 production, RP105-MD-1 expression did not (FIG. 3a). It should be noted that these data are consistent with data generated previously using Ba/F3 cells: even in B cell lines, no direct role for RP105 as a signaling receptor for LPS was shown²⁵. We confirmed that in vitro overexpression of TLR4-MD-2 in HEK293 cells led to an increase in baseline IL-8 production in the absence of stimulation (FIG. 3a).

The effects of RP105-MD-1 expression on LPS-driven TLR4 signaling were also examined. Notably, RP105 expression inhibited TLR4-driven IL-8 production by HEK293 cells in a dose-dependent manner (FIG. 3b). Further, RP105-mediated inhibition of LPS-driven IL-8 production was associated with inhibition of LPS-driven NF-κB transactivation (FIG. 3c), suggesting that modulation of IL-8 production by RP105 was upstream of NF-κB activation.

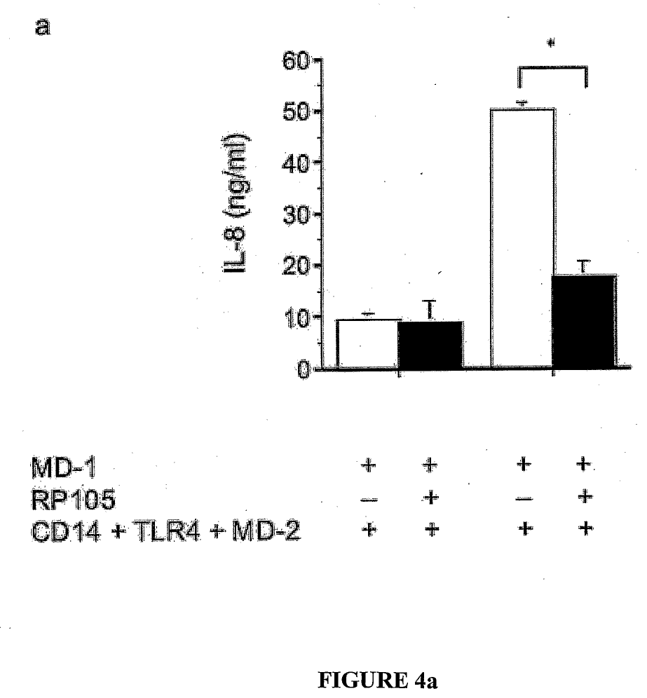

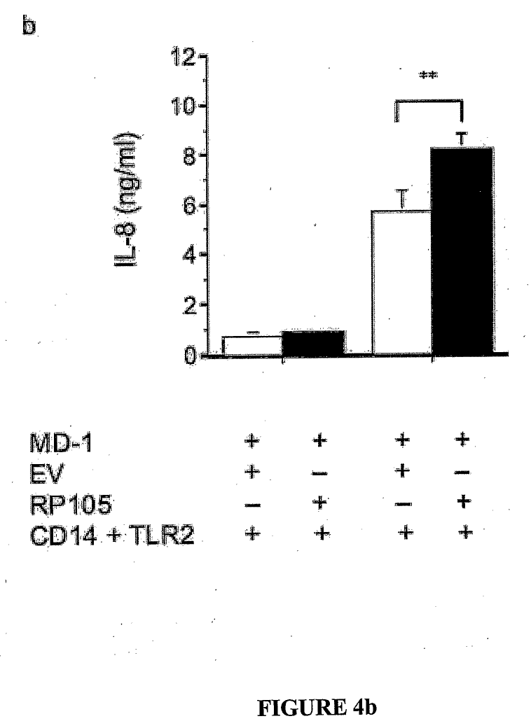

The specificity of RP105-mediated suppression of proinflammatory signaling was subsequently examined. RP105 did not inhibit IL-1- or TLR2-driven IL-8 production (FIG. 4). Indeed, RP105 overexpression led to variable augmentation of TLR2-mediated IL-8 production in some experiments (shown in FIG. 4b). Thus, RP105-mediated suppression exhibits specificity among the TLR/IL-1R family of receptors.

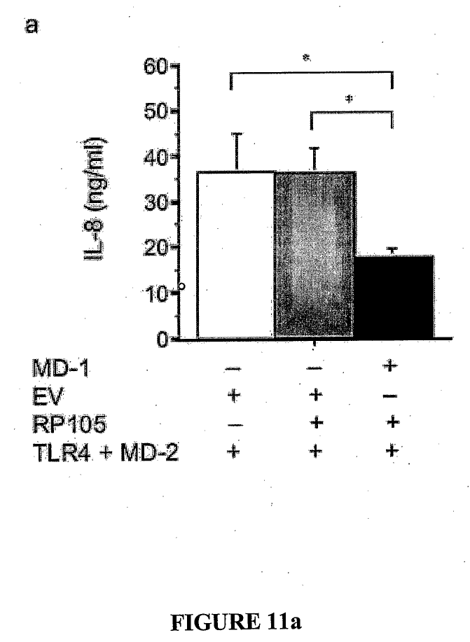

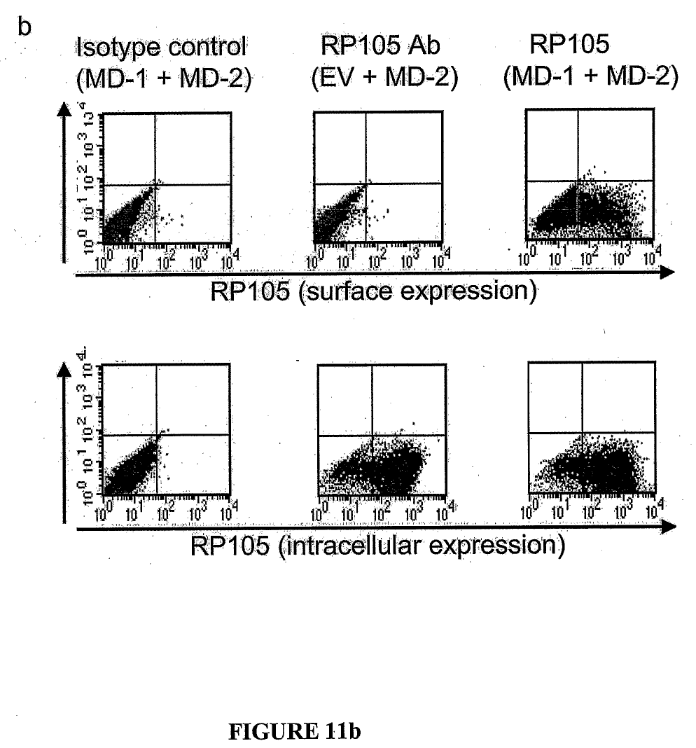

The necessity for MD-1 expression in this system was formally examined. In the absence of MD-1 co-expression, RP105 did not inhibit TLR4 signaling (FIG. 11a). Consistent with the previous observation that MD-1 expression is required for surface expression of RP105¹³, RP105 failed to be detected on cells in the absence of MD-1 co-expression (FIG. 11b). Thus, RP105-MD-1 is a specific inhibitor of TLR4 signaling in HEK293 cells.

Suppression by the RP105 Extracellular Domain

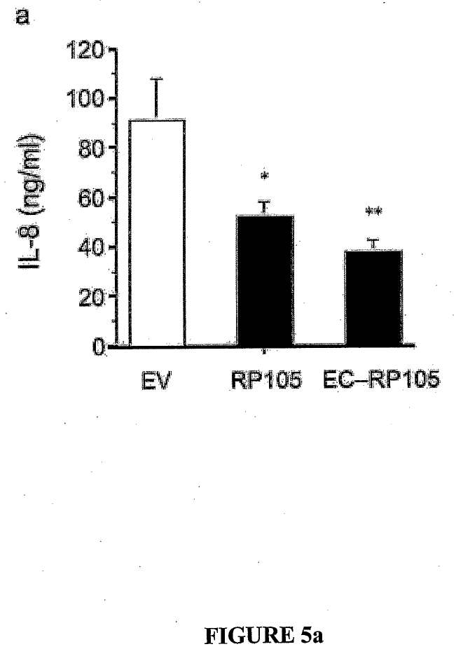

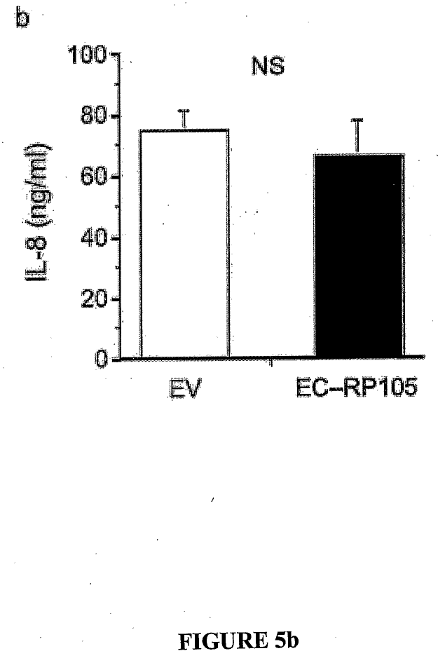

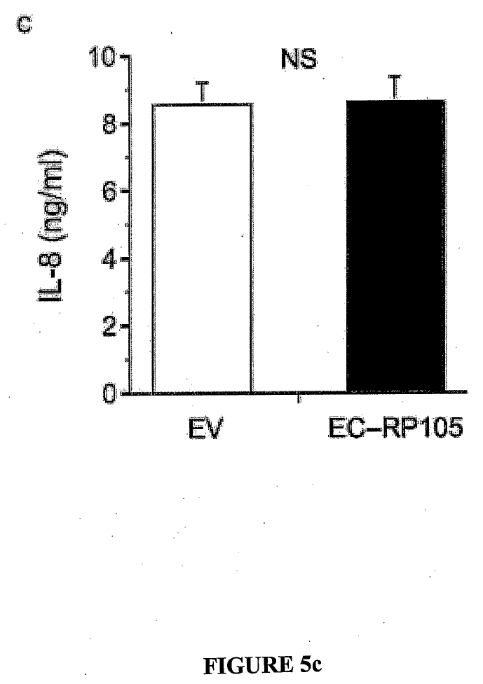

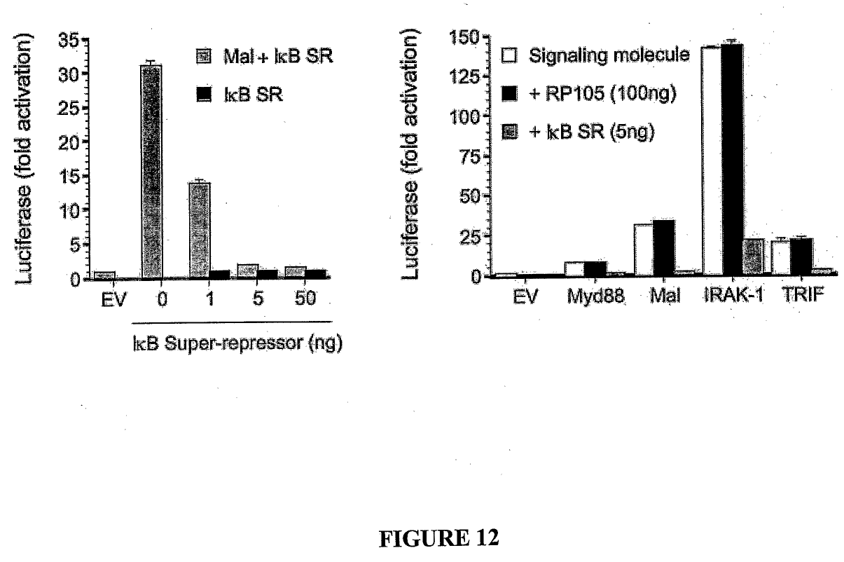

Antibodies to RP105 can induce signaling events and proliferation in B cells, although there is no evidence that RP105 signals directly^{6,10,25-27}. Use of such antibodies in human primary monocyte/macrophages did not induce a measurable signal (cytokine production; data not shown), an observation that suggested that the mechanism of suppressive action of RP105 might be independent of any putative signaling through its intracellular tail. Consistent with this, RP105 did not inhibit NF-1-κB transactivation induced through the overexpression of TLR4 signaling molecules (FIG. 12)²⁸. A soluble mutant of RP105, lacking the transmembrane and intracellular domains, was thus constructed. Initial analysis by immunoprecipitation revealed that the RP105 extracellular domain protein was secreted into the medium of transfected cells (data not shown). Mechanistic analysis revealed that co-expression of MD-1 and the extracellular portion of RP105 is sufficient to effect RP105-mediated suppression of TLR4 signaling (FIG. 5a). Identical to findings with the full-length construct, suppression of signaling by the extracellular domain of RP105 had considerable specificity, failing to inhibit TLR2 or IL-1R signaling in HEK293 cells (FIGS. 5b and 5c). Thus, inhibition of TLR4 signaling is mediated by the extracellular domain of RP105.

Direct Interaction of RP105-MD-1 with TLR4-MD-2

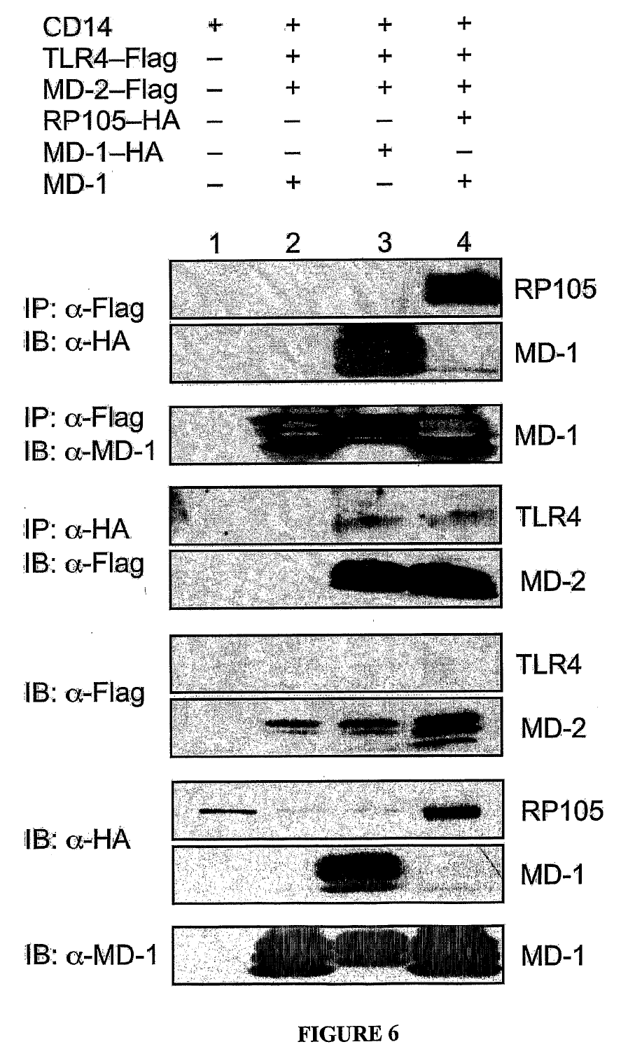

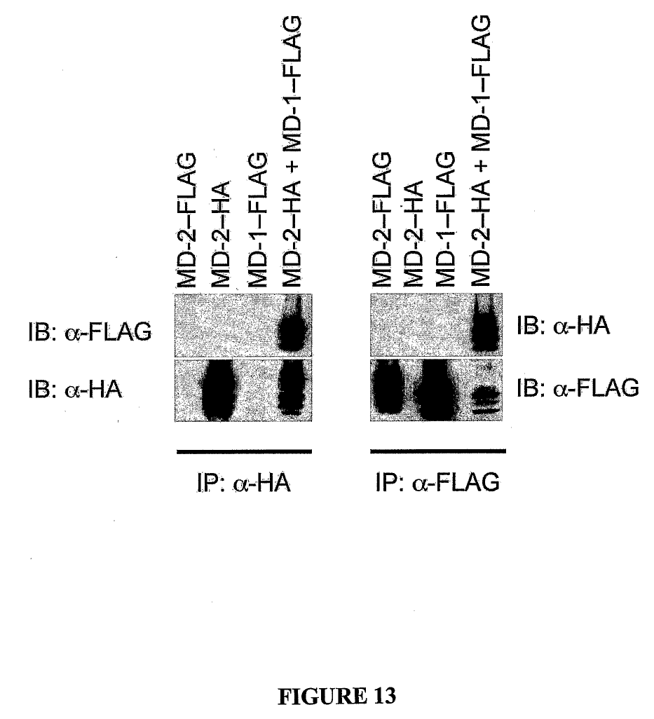

The extracellular localization of the inhibitory effects of the RP105 suggested a mechanistic model involving direct interactions between the RP105-MD-1 and TLR4-MD-2 complexes. Co-immunoprecipitation techniques were used to probe the association of these complexes. These complexes co-immunoprecipitate bi-directionally, demonstrating physical association between TLR4-MD-2 and RP105-MD-1 (FIG. 6). As MD-1 failed to associate with TLR4 in the absence of MD-2 (data not shown), the ability of MD-1 to associate with TLR4-MD-2 (FIG. 6, lanes 2 and 3) suggested the possibility that MD-1 and MD-2 interact directly. Indeed, immunoprecipitation analysis revealed the presence of such interactions (FIG. 13).

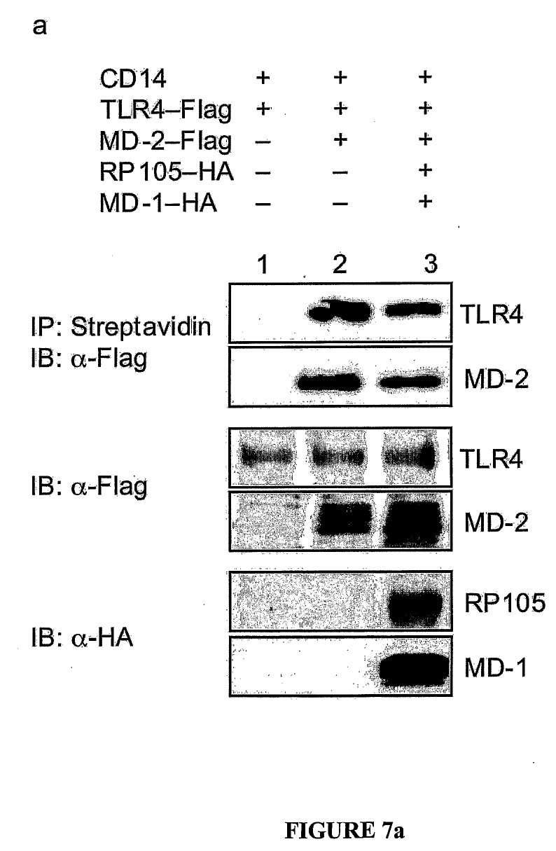

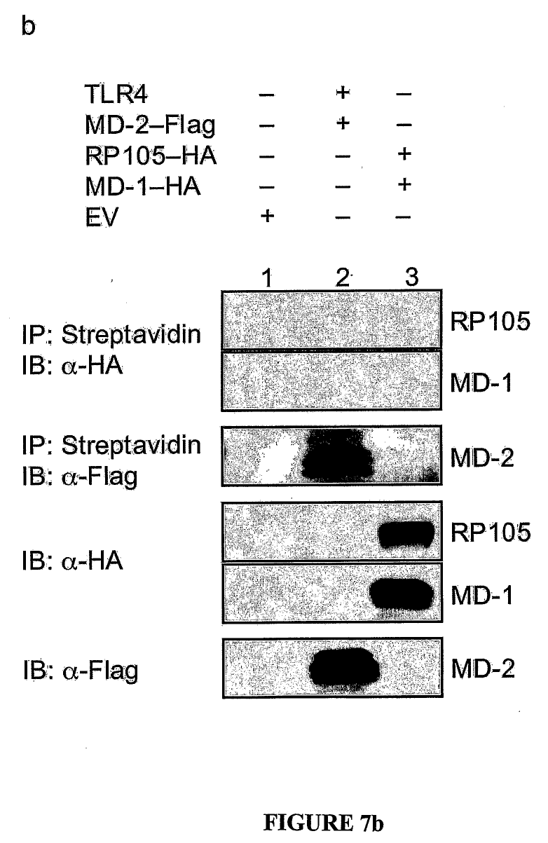

Biotin-labeled LPS has been used to demonstrate that LPS binds directly to MD-2, leading to the association of LPS/MD-2 complexes with TLR4, and to TLR4 signaling²⁹. In replication of these data, incubation of biotinylated LPS with TLR4-expressing HE 93 cells allowed for precipitation of TLR4 (and MD-2) only in the presence of MD-2 expression (FIG. 7a). Of note, co-expression of RP105-MD-1 in this system inhibited LPS-TLR4-MD-2 complex formation (FIG. 7a), providing direct evidence that RP 05-MD-1 inhibits LPS signaling complex formation. The fact that, consistent with previously reported data³⁰, no direct interactions between LPS and RP105-MD-1 were shown in this system (FIG. 7b) indicates that RP105-MD-1-mediated interference with LPS signaling complex formation is not due to RP105-MD-1 acting as a molecular sink for LPS. Thus, RP105-MD-1 interacts directly with the TLR4 signaling complex, inhibiting its ability to bind microbial ligand.

RP105 is a Physiological Regulator of Cytokine Production

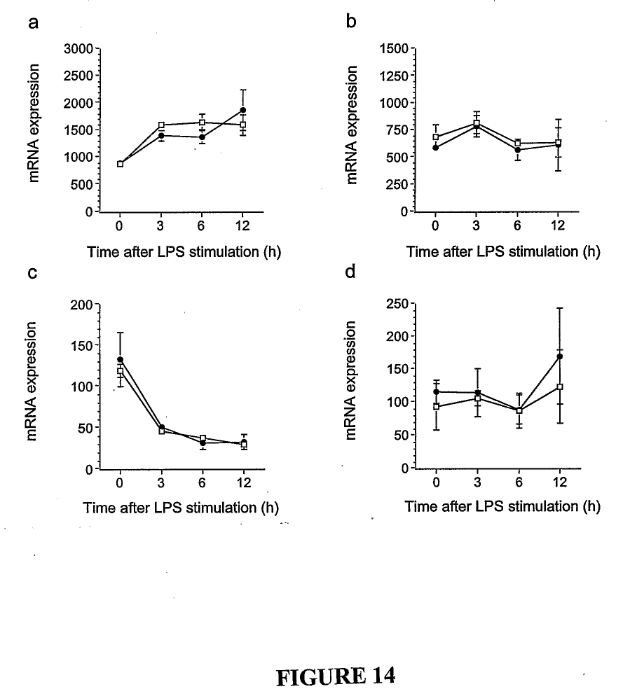

Although expression studies in cell lines have considerable utility, they also have obvious drawbacks; principally, that overexpression may drive non-physiological interactions and processes. To confirm and extend our findings in transfected cell lines, we thus examined RP105-deficient mice, generated by targeted disruption of exon 3 of RP105²⁵ and backcrossed for more than 10 generations onto the C57BL/6 background. Bone marrow-derived DC were generated by standard techniques³¹ from age-matched RP105-deficient mice and wild-type littermate controls. DC generated from RP105-deficient mice appeared to be phenotypically normal. In particular, FACS analysis revealed no differences between DC generated from RP105-sufficient and -deficient mice in baseline expression of CD14, TLR4, CD83, CD11c, CD80, CD86 or major histocompatibility complex (MHC) class II (data not shown). Further, RT-PCR analysis revealed no differences in expression of any of the TLRs, MD-1, MD-2, or the TLR pathway inhibitors IRAK-M, single immunoglobulin IL-1R-related protein (SIGIRR), ST2, Tollip or SOCS1 in such DC (FIG. 14; and data not shown).

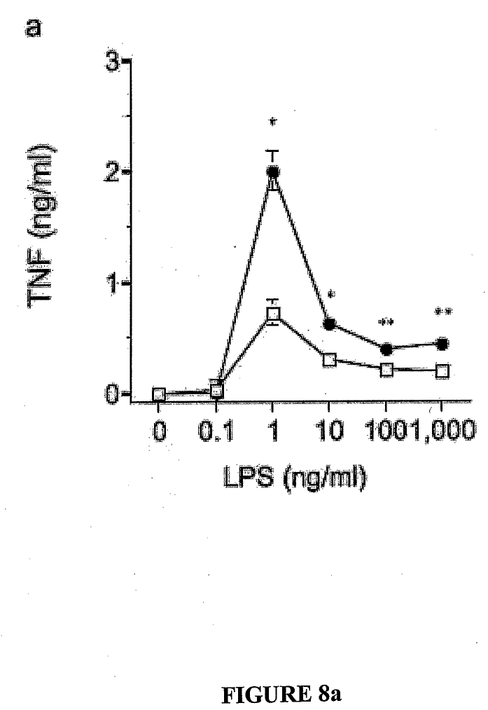

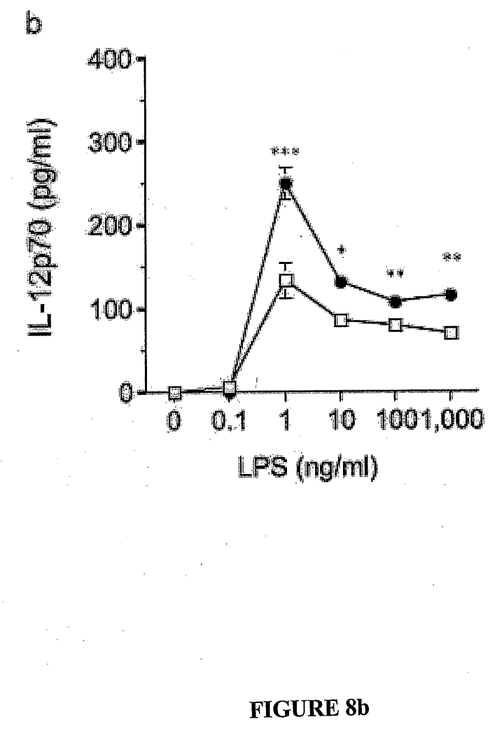

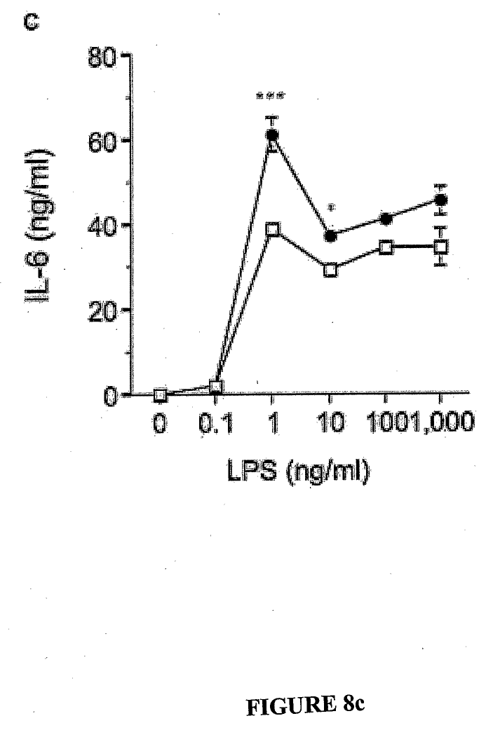

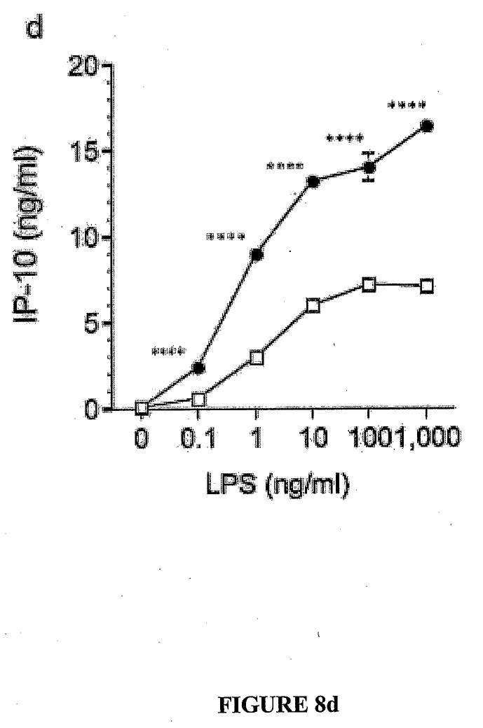

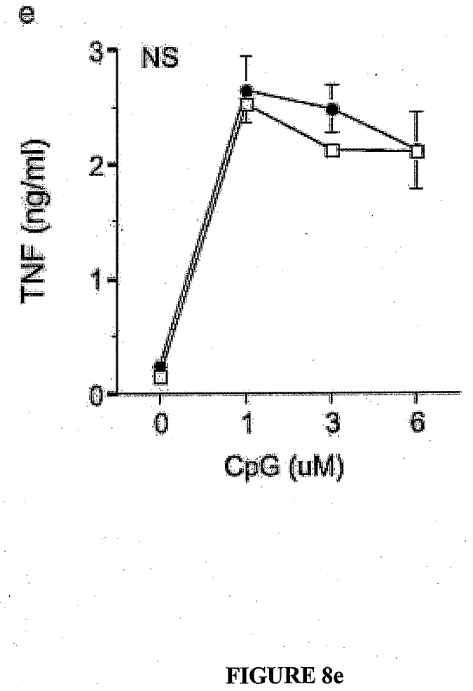

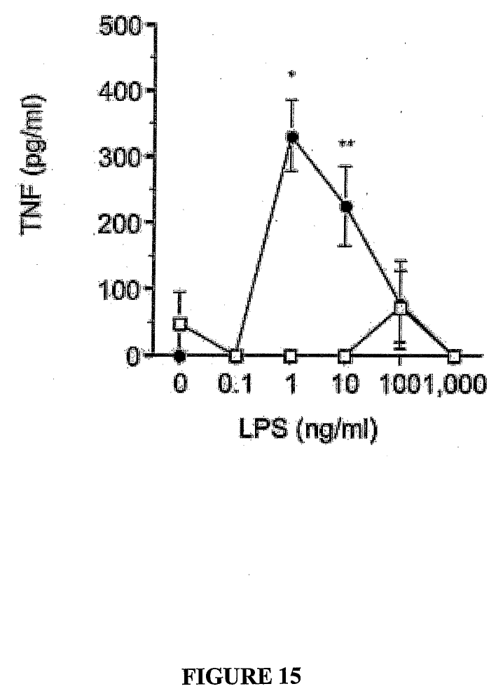

DC from RP105-deficient mice produced significantly higher concentrations of proinflammatory cytokines after stimulation with purified E. coli LPS than did DC from wild-type controls (FIG. 8). Thus, RP105-mediated suppression of TLR4 signaling is not merely an overexpression artifact in cell lines. Increased TLR4-driven cytokine production occurred for tumor necrosis factor (TNF), IL-112p70 and IL-6 (FIG. 8a-c), cytokines whose TLR4-driven production occurs through the signaling adaptor proteins Mal-MyD88, as well as for IP-10 (FIG. 8d), a chemokine whose TLR4-driven production occurs through a complementary signaling pathway mediated by the TRIF-TRAM adaptor proteins. RP105-mediated modulation of cytokine production by DC also exhibited specificity; for example, CpG (TLR9)-driven cytokine production was unaltered in DC generated from RP105-deficient mice (FIG. 8e). RP105-mediated modulation of proinflammatory cytokine production was also observed in macrophages; resident peritoneal macrophages from RP105-deficient mice produced significantly higher concentrations of TNF after stimulation with purified E. coli LPS than did macrophages from wild-type controls (FIG. 15).

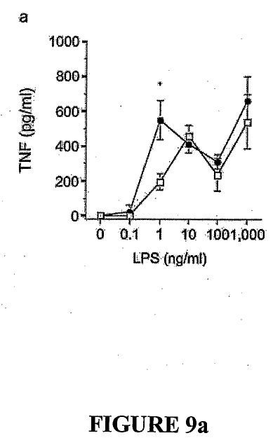

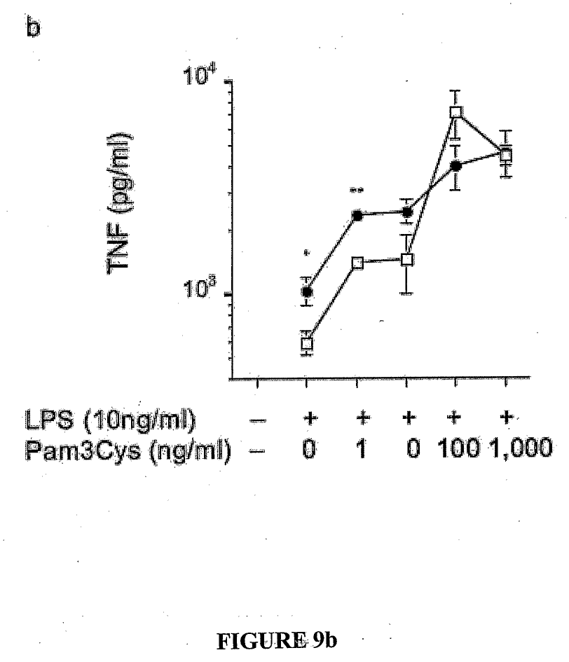

The biphasic dose-response for TNF, IL-12p70 and IL-6 production observed in DC from both wild-type and knockout mice (FIG. 8) was examined further. Flow cytometric analysis revealed that the reduced cytokine production at higher doses of LPS was not associated with greater DC apoptosis (data not shown). Commercial LPS preparations are typically contaminated with lipopeptide ligands for TLR³². Such preparations of LPS continue to drive increasing TNF production at higher doses (FIG. 9a), the very doses at which such preparations drive IL-8 production by HEK293 cells transfected with TLR2 (data not shown). Notably, stimulation of DC with these higher doses of commercial LPS preparations led to similar amounts of TNF production by DC from RP105-deficient and wild-type mice (FIG. 9a). Indeed, stimulation with combinations of purified TLR4 and TLR2 agonists revealed that TLR2 agonists are able to effect functional reversal of RP105-mediated inhibition of TLR4 signaling (FIG. 9b). These data indicate that, as with many biological agonistic responses, the response to a pure TLR4 ligand is biphasic, something modifiable by secondary agonists; and that signaling through other TLRs can overcome RP105-mediated modulation of TLR4 signaling.

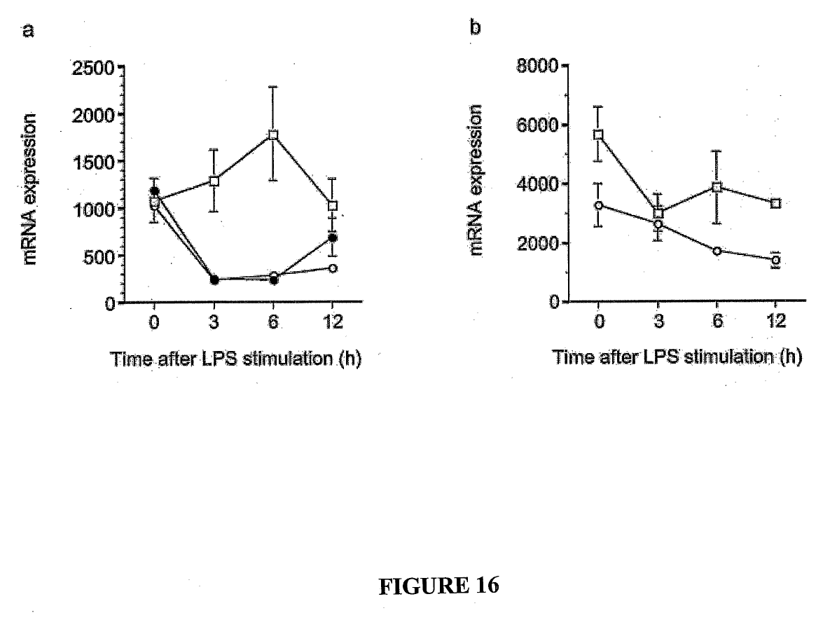

Other modulators of TLR signaling have, themselves, been found to be regulated by TLR signaling³³. Quantitative RT-PCR was used to examine RP105 expression after LPS stimulation of murine DC. TLR4 signaling induced transient upregulation of RP105 mRNA expression, along with the previously-described downregulation of TLR4 mRNA expression³⁴, something unaltered by a lack of RP105 expression in such cells (FIG. 16a). Despite these findings, preliminary experiments have not revealed a role for RP105 in endotoxin tolerance (data not shown). Of interest, LPS stimulation of human DC induced coordinate down-regulation of both TLR4 and RP105 (FIG. 16b), adding to growing findings of divergent regulation of TLRs in humans and mice³⁵.

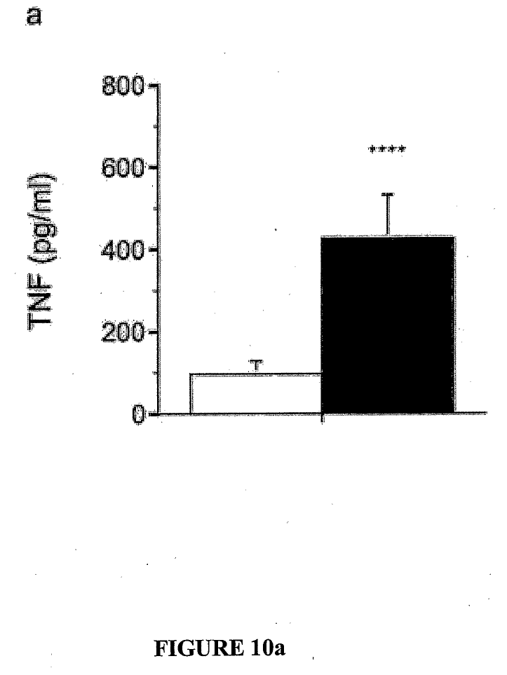

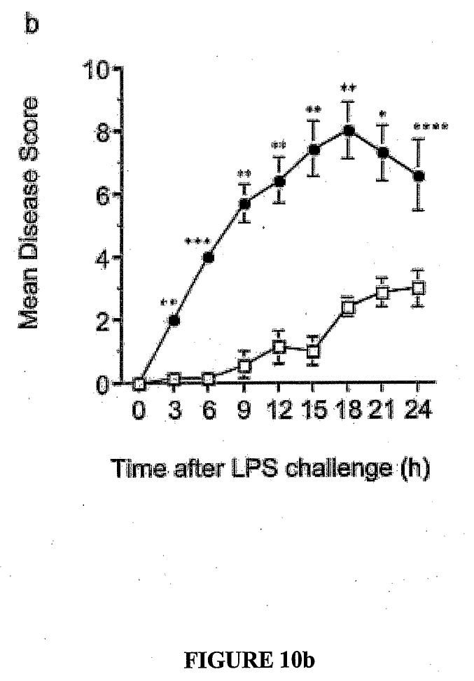

Finally, LPS-driven in vivo responses were compared in RP105-deficient and wild-type mice. Notably, RP105-deficient mice produced significantly more TNF in response to low dose intraperitoneal challenge with E. coli LPS (FIG. 10a). Further, high dose LPS challenge led to significant acceleration and amplification of endotoxicity in RP105-deficient mice (FIG. 10b). Thus, RP105 is a physiological regulator of responses to LPS, in primary myeloid cells in vitro as well as in vivo.

The studies reported here show that RP105, a TLR4 homolog whose expression mirrors that of TLR4 on antigen presenting cells, is a negative regulator of TLR4 signaling. RP105 specifically inhibited TLR4 signaling when co-expressed in HEK293 cells. Furthermore, RP105 was a physiological regulator of endotoxin-driven TLR4 signaling in DC, as well as of endotoxicity in vivo.

Although the activation of proinflammatory responses through TLRs is critical for host defense, excessive or inappropriate inflammation can itself be maladaptive. RP105 joins a growing list of molecules that can inhibit TLR signaling. Regulation of TLR expression provides one point of control², as does the complex phenomenon of endotoxin tolerance³⁶. Several direct, negative regulators of TLR signaling have also been found, including SIGIRR³³, IRAK-M³⁷, MyD88s³⁸, Tollip³⁹, ST²⁸, NOD2⁴⁰, and Triad3A⁴¹. Among this list, RP105 stands out for its specificity for inhibition of TLR4 signaling.

The description fully satisfies the objects, aspects and advantages set forth. While the invention has been set forth in conjunction with specific embodiments thereof, it is evident that many alternatives, modifications, and variations will be apparent to those skilled in the art in the light of the foregoing description. Accordingly, it is intended to embrace all such alternatives, modifications, and variations which fall within the spirit and scope of the following claims.

### REFERENCES

1. Medzhitov, R. & Janeway, C., Jr. Innate immune recognition: mechanisms and pathways. Immunol Rev 173, 89-97 (2000).
2. Takeda, K., Kaisho, T. & Akira, S. Toll-like receptors. Annu Rev Immunol 21, 335-76. (2003).
3. Beutler, B. & Rietschel, E. T. Innate immune sensing and its roots: the story of endotoxin. Nat Rev Immunol 3, 169-76 (2003).
4. Ardizzone, S. & Porro, G. B. Inflammatory bowel disease: new insights into pathogenesis and treatment. J Intern Med 252, 475-96 (2002).
5. Bingham, C. O., 3rd. The pathogenesis of rheumatoid arthritis: pivotal cytokines involved in bone degradation and inflammation. J Rheumatol Suppl 65, 3-9 (2002).
6. Miyake, K., Yamashita, Y., Hitoshi, Y., Takatsu, K. & Kimoto, M. Murine B cell proliferation and protection from apoptosis with an antibody against a 105-kD molecule: unresponsiveness of X-linked immunodeficient B cells. J Exp Med 180, 1217-24 (1994).
7. Miyake, K., Yamashita, Y., Ogata, M., Sudo, T. & Kimoto, M. RP105, a novel B cell surface molecule implicated in B cell activation, is a member of the leucine-rich repeat protein family. J Immunol 154, 3333-40 (1995).
8. Miura, Y. et al. Molecular cloning of a human RP105 homologue and chromosomal localization of the mouse and human RP105 genes (Ly64 and LY64). Genomics 38, 299-304 (1996).
9. Fugier-Vivier, I. et al. Molecular cloning of human RP105. Eur J Immunol 27, 1824-7 (1997).
10. Roshak, A. K. et al. Anti-human RP105 sera induces lymphocyte proliferation. J Leukoc Biol 65, 43-9 (1999).
11. Shimazu, R. et al. MD-2, a Molecule that Confers Lipopolysaccharide Responsiveness on Toll-like Receptor 4. J Exp Med 189, 1777-1782 (1999).
12. Miyake, K. et al. Mouse MD-1, a molecule that is physically associated with RP105 and positively regulates its expression. J Immunol 161, 1348-53 (1998).
13. Miura, Y. et al. RP105 is associated with MD-1 and transmits an activation signal in human B cells. Blood 92, 2815-22 (1998).
14. Nagai, Y. et al. Requirement for MD-1 in cell surface expression of RP105/CD180 and B-cell responsiveness to lipopolysaccharide. Blood 99, 1699-705 (2002).
15. Schneider, D. S., Hudson, K. L., Lin, T. Y. & Anderson, K. V. Dominant and recessive mutations define functional domains of Toll, a transmembrane protein required for dorsal-ventral polarity in the Drosophila embryo. Genes Dev 5, 797-807 (1991).
16. Winans, K. A. & Hashimoto, C. Ventralization of the Drosophila embryo by deletion of extracellular leucine-rich repeats in the Toll protein. Mol Biol Cell 6, 587-96 (1995).
17. Medzhitov, R., Preston-Hurlburt, P. & Janeway, C. A., Jr. A human homologue of the Drosophila Toll protein signals activation of adaptive immunity. Nature 388, 394-7 (1997).
18. Vogel, S. N. et al. Cutting edge: functional characterization of the effect of the C3H/HeJ defect in mice that lack an lpsn gene: In vivo evidence for a dominant negative mutation. J Immunol 162, 5666-70 (1999).
19. Ronni, T. et al. Common interaction surfaces of the toll-like receptor 4 cytoplasmic domain stimulate multiple nuclear targets. Mol Cell Biol 23, 2543-55 (2003).
20. Jarrossay, D., Napolitani, G., Colonna, M., Sallusto, F. & Lanzavecchia, A. Specialization and complementarity in microbial molecule recognition by human myeloid and plasmacytoid dendritic cells. Eur J Immunol 31, 3388-93 (2001).
21. Kadowaki, N. et al. Subsets of human dendritic cell precursors express different toll-like receptors and respond to different microbial antigens. J Exp Med 194, 863-9 (2001).
22. Krug, A. et al. Toll-like receptor expression reveals CpG DNA as a unique microbial stimulus for plasmacytoid dendritic cells which synergizes with CD40 ligand to induce high amounts of IL-12. Eur J Immunol 31, 3026-37. (2001).
23. Latz, E. et al. Lipopolysaccharide rapidly traffics to and from the Golgi apparatus with the toll-like receptor 4-MD-2-CD14 complex in a process that is distinct from the initiation of signal transduction. J Biol Chem 277, 47834-43 (2002).
24. Kurt-Jones, E. A. et al. Pattern recognition receptors TLR4 and CD14 mediate response to respiratory syncytial virus. Nat Immunol 1, 398-401. (2000).
25. Ogata, H. et al. The toll-like receptor protein RP105 regulates lipopolysaccharide signaling in B cells. J Exp Med 192, 23-9 (2000).
26. Chan, V. W. et al. The molecular mechanism of B cell activation by toll-like receptor protein RP-105. J Exp Med 188, 93-101 (1998).
27. Yazawa, N. et al. CD19 regulates innate immunity by the toll-like receptor RP105 signaling in B lymphocytes. Blood 102, 1374-80 (2003).
28. Brint, E. K. et al. ST2 is an inhibitor of interleukin 1 receptor and Toll-like receptor 4 signaling and maintains endotoxin tolerance. Nat Immunol 5, 373-9 (2004).
29. Visintin, A., Latz, E., Monks, B. G., Espevik, T. & Golenbock, D. T. Lysines 128 and 132 enable lipopolysaccharide binding to MD-2, leading to Toll-like receptor-4 aggregation and signal transduction. J Biol Chem 278, 48313-20 (2003).
30. Tsuneyoshi, N. et al. The functional and structural properties of MD-2 required for lipopolysaccharide binding are absent in MD-1. J Immunol 174, 340-4 (2005).
31. Belkaid, Y., Piccirillo, C. A., Mendez, S., Shevach, E. M. & Sacks, D. L. CD4+ CD25+ regulatory T cells control Leishmania major persistence and immunity. Nature 420, 502-7. (2002).
32. Hirschfeld, M., Ma, Y., Weis, J. H., Vogel, S. N. & Weis, J. J. Cutting edge: repurification of lipopolysaccharide eliminates signaling through both human and murine toll-like receptor 2. J Immunol 165, 618-22 (2000).
33. Wald, D. et al. SIGIRR, a negative regulator of Toll-like receptor-interleukin 1 receptor signaling. Nat Immunol 4, 920-7 (2003).
34. Poltorak, A. et al. Defective LPS signaling in C3H/HeJ and C57BL/10ScCr mice: mutations in Tlr4 gene. Science 282, 2085-8 (1998).
35. Rehli, M. Of mice and men: species variations of Toll-like receptor expression. Trends Immunol 23, 375-8 (2002).
36. Cross, A. S. Endotoxin tolerance-current concepts in historical perspective. J Endotoxin Res 8, 83-98. (2002).
37. Kobayashi, K. et al. IRAK-M is a negative regulator of Toll-like receptor signaling. Cell 110, 191-202. (2002).
38. Burns, K. et al. Inhibition of interleukin 1 receptor/Toll-like receptor signaling through the alternatively spliced, short form of MyD88 is due to its failure to recruit IRAK-4. J Exp Med 197, 263-8 (2003).
39. Zhang, G. & Ghosh, S. Negative regulation of toll-like receptor-mediated signaling by Tollip. J Biol Chem 277, 7059-65 (2002).
40. Watanabe, T., Kitani, A., Murray, P. J. & Strober, W. NOD2 is a negative regulator of Toll-like receptor 2-mediated T helper type 1 responses. Nat Immunol 5, 800-8 (2004).
41. Chuang, T. H. & Ulevitch, R. J. Triad3A, an E3 ubiquitin-protein ligase regulating Toll-like receptors. Nat Immunol 5, 495-502 (2004).
42. Akashi, S. et al. Cutting edge: cell surface expression and lipopolysaccharide signaling via the toll-like receptor 4-MD-2 complex on mouse peritoneal macrophages. J Immunol 164, 3471-5. (2000).
43. Vogel, S. N., Fitzgerald, K. A. & Fenton, M. J. TLRs: Differential adapter utilization by Toll-like receptors mediates TLR-specific patterns of gene expression. Mol Interventions 3, 466-477 (2003).
44. McIntire, F. C., Sievert, H. W., Barlow, G. H., Finley, R. A. & Lee, A. Y. Chemical, physical, biological properties of a lipopolysaccharide from Escherichia coli K-235. Biochemistry 6, 2363-72 (1967).
45. Kurt-Jones, E. A. et al. Role of toll-like receptor 2 (TLR2) in neutrophil activation: GM-CSF enhances TLR2 expression and TLR2-mediated interleukin 8 responses in neutrophils. Blood 100, 1860-8 (2002).
46. Acred, P. et al. Guidelines for the welfare of animals in rodent protection tests: A report from the rodent protection test working party. Lab Anim 28, 13-18 (1994).
47. Karp, C. L. et al. Mechanism of suppression of cell-mediated immunity by measles virus. Science 273, 228-31 (1996).
48. Atabani, S. F. et al. Natural measles causes prolonged suppression of interleukin-12 production. J Infect Dis 184, 1-9 (2001).
49. Jeanmougin, F., Thompson, J. D., Gouy, M., Higgins, D. G. & Gibson, T. J. Multiple sequence alignment with Clustal X. Trends Biochem Sci 23, 403-5 (1998).
50. Devereux, J., Haeberli, P. & Smithies, O. A comprehensive set of sequence analysis programs for the VAX. Nucleic Acids Res 12, 387-95 (1984).

What is claimed is:

1. A method of using of an activator of RP105 to prepare a medicament to suppress an immune response.

2. The method according to claim 1 wherein the medicament is used to prevent or treat an autoimmune disease.

3. A use according to claims 1 or 2 wherein the activator of RP105 is an oligonucleotide that activates the expression of RP105.

4. A use according to any one of claims 1 or 2 wherein the activator of RP105 is an antibody that binds to RP105.

5. A use according to any one of claims 1 to 4 wherein the medicament further comprises an ORP105 protein or a nucleic acid molecule encoding an RP105 protein.

6. A use of an RP105 protein or a nucleic acid sequence encoding an RP105 protein to prepare a medicament to inhibit an immune response.

7. A pharmaceutical composition for use in suppressing an immune response comprising an activator of RP105 in admixture with a suitable diluent or carrier.

8. A composition according to claim 7 wherein the activator is an antibody that binds RP105.

9. A composition according to claim 7 wherein the activator is an oligonucleotide that is complimentary to a nucleic acid sequence from an RP105 gene.

10. A pharmaceutical composition for use in preventing immune suppression comprising an RP105 protein or a nucleic acid encoding an RP105 protein in admixture with a suitable diluent or carrier.

11. A method of identifying substances that can activate RP105, comprising the steps of: (a) reacting RP105 and a test substance, under conditions which allow for formation of a complex between the RP105 and the test substance, and (b) assaying for complexes of RP105 and the test substance, for free substance or for activation of RP105.

12. A method of identifying substances that can activate RP105, comprising the steps of: (a) reacting RP105 and a test substance, under conditions which allow for the activation of the RP105, and (b) assaying for activation of RP105.

13. A method of identifying substances that can activate RP105, comprising the steps of: (a) reacting RP105 and a test substance, under conditions which allow for the activation of the RP105, and (b) assaying for the inhibition of the activity or expression level of TLR4.

14. A method of using a compound identified according to claim 11 to prepare a medicament to modulate an inflammatory or immune response.

15. A method of using a compound identified according to claim 12 to prepare a medicament to suppress an inflammatory or immune response.

16. A method of using a compound identified according to claim 13 to prepare a medicament to suppress an inflammatory or immune response.

17. A method of treating a TLR-4 mediated disease or pathology in a subject comprising administering a pharmaceutically effective amount of an activator of RP105 to the subject.

18. The method according to claim 17, wherein the TLR-4 mediated disease or pathology is selected from the group consisting of psoriasis, atopic dermatitis, asthma, COPD, adult respiratory disease, arthritis, inflammatory bowel disease, Crohn's disease, ulcerative colitis, septic shock, endotoxic shock, gram negative sepsis, toxic shock syndrome, stroke, cardiac and renal reperfusion injury, glomerulonephritis, thrombosis, Alzheimer's disease, graft vs. host reaction, allograft rejections, malaria, acute respiratory distress syndrome, delayed type hypersensitivity reaction, atherosclerosis, cerebral and cardiac ischemia, osteoarthritis, multiple sclerosis, restinosis, angiogenesis, osteoporosis, gingivitis, respiratory viruses, herpes viruses, hepatitis viruses, HIV, Kaposi's sarcoma associated virus, meningitis, cystic fibrosis, pre-term labor, cough, pruritis, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, angiogenic ocular disease, ocular inflammation, retinopathy of prematurity, diabetic retinopathy, macular degeneration with the wet type preferred and corneal neovascularization, polymyositis, vasculitis, acne, gastric and duodenal ulcers, celiac disease, esophagitis, glossitis, airflow obstruction, airway hyperresponsiveness, bronchiectasis, bronchiolitis, bronchiolitis obliterans, chronic bronchitis, cor pulmonae, cough, dyspnea, emphysema, hypercapnea, hyperinflation, hypoxemia, hyperoxia-induced inflammations, hypoxia, surgical lung volume reduction, pulmonary fibrosis, pulmonary hypertension, right ventricular hypertrophy, peritonitis associated with continuous ambulatory peritoneal dialysis (CAPD), granulocytic ehrlichiosis, sarcoidosis, small airway disease, ventilation-perfusion mismatching, wheeze, colds, gout, alcoholic liver disease, lupus, burn therapy, periodontitis and early transplantation.

19. The method according to claim 17, wherein the compounds of the present invention are administered in conjunction with one or more drugs, agents or therapeutics selected from the group consisting of: glucocorticoids, 5-lipoxygenase inhibitors, β-2 adrenoreceptor agonists, muscarinic M1 and M3 antagonists, muscarinic M2 agonists, NK3 antagonists, LTB4 antagonists, cysteinyl leukotriene antagonists, bronchodilators, PDE4 inhibitors, PDE inhibitors, elastase inhibitors, MMP inhibitors, phospholipase A2 inhibitors, phospholipase D inhibitors, histamine H1 antagonists, histamine H3 antagonists, dopamine agonists, adenosine A2 agonists, NK1 and NK2 antagonists, GABA-b agonists, nociceptin agonists, expectorants, mucolytic agents, decongestants, antioxidants, anti-IL-8 anti-bodies, anti-IL-5 antibodies, anti-IgE antibodies, anti-TNF antibodies, IL-10, adhesion molecule inhibitors, and growth hormones.

20. The method according to claim 17, wherein the compounds of the present invention are administered in conjunction with one or more therapeutic steroids.

21. The method according to claim 17, wherein the one or more therapeutic steroids are selected from the group consisting of corticoids, glucocorticoids, dexamethasone, prednisone, prednisalone, and betamethasone.

22. A method of treating a condition associated with cytokine production in a subject comprising administering a pharmaceutically effective amount of an activator of RP105 to the subject.

23. A method of using an activator of RP105 in the manufacture of a medicament for the treatment of a condition associated with cytokine production, wherein the condition is rheumatoid arthritis or a condition resulting from an infection.

24. The method according to claim 22 wherein the condition is a tumor necrosis factor (TNF) associated condition.

25. The method according to claim 24 wherein the TNF is TNF-α.

26. The method according to claim 22 wherein the condition is an interleukin-1 (IL-1) associated condition.

27. The method according to claim 26 wherein the IL-1 is IL-1.

28. The method according to claim 22 wherein the condition is sepsis.

29. The method according to claim 22 wherein the condition is septic shock.

30. The method according to claim 22 wherein the condition is systemic inflammatory response syndrome

31. The method according to claim 17 or 22 wherein the condition is induced by a Toll Related Receptor (TRR) ligand.

32. The method according to claim 17 or 22 wherein the condition is induced by lipopolysaccharide (LPS).

33. The method according to claim 17 or 22 wherein the condition is induced by Gram-negative bacteria.

34. The method according to claim 17 or 22 wherein the activator is specific for RP105.

35. The method according to claim 17 or 22 wherein the activator is a chemical activator.

36. The method according to claim 17 or 22 wherein the activator is an antibody or a fragment thereof that is capable of binding specifically to RP105 or a fragment thereof.

37. The method according to claim 17 or 22 wherein the activator is a nucleic acid capable of increasing the expression of RP105.

38. A method for identifying an activator of RP105, which activator is suitable for use in the treatment of a condition associated with cytokine production wherein the condition s marked by pathogenic inflammatory or immune responses, the method comprising: (a) providing, as a first component, a cell capable of TLR4 or TLR ligand-induced cytokine production; (b) providing, as a second component, a TLR ligand or TLR4 activator; (c) contacting the first and second components in the presence of a test agent; (d) determining whether the test agent is able to increase RP105-mediated downregulation of TLR4 activity; thereby to determine whether the test agent acts as an inhibitor of cytokine production.

39. The method according to claim 38 further comprising the step of determining whether the activator is specific for RP105.

40. The activator of TLR4-induced expression of cytokines identified or identifiable by a method as defined in claim 38.

41. A pharmaceutical formulation comprising a pharmaceutically acceptable carrier and an activator of RP105 identifiable by a method according to claim 38.

42. A method of using an activator of RP105 to study the inhibition of sepsis and/or septic shock caused by a TLR4, in vitro.


SEQUENCE LISTING (21 sequences)

SEQ ID NO: 1 - DNA, 21 bp, Artificial Sequence
  Primer RP105
agtctcctccccatcttgtcc

SEQ ID NO: 2 - DNA, 21 bp, Artificial Sequence
  Primer RP105
gatagcgtcacatcggagagc

SEQ ID NO: 3 - DNA, 20 bp, Artificial Sequence
  Primer TLR4
catccaggaaggcttccaca

SEQ ID NO: 4 - DNA, 20 bp, Artificial Sequence
  Primer TLR4
ggcgatacaattccacctgc

SEQ ID NO: 5 - DNA, 21 bp, Artificial Sequence
  Primer IRAK-M
gagaattgctctggtcctggg

SEQ ID NO: 6 - DNA, 20 bp, Artificial Sequence
  Primer IRAK-M
cacctcaagtgggaagctgg

SEQ ID NO: 7 - DNA, 20 bp, Artificial Sequence
  Primer SIGIRR
ggcccctaatttcctttccc

SEQ ID NO: 8 - DNA, 20 bp, Artificial Sequence
  Primer SIGIRR
catggaggctgaagtggctt

SEQ ID NO: 9 - DNA, 19 bp, Artificial Sequence
  Primer Tollip
tggacccacatcaccatcc

SEQ ID NO: 10 - DNA, 20 bp, Artificial Sequence
  Primer Tollip
gttggcatcaggaccacagg

SEQ ID NO: 11 - DNA, 22 bp, Artificial Sequence
  Primer ST2
ggctctcacttcttggctgatg

SEQ ID NO: 12 - DNA, 22 bp, Artificial Sequence
  Primer ST2
gccagacagtcatattccaggg

SEQ ID NO: 13 - DNA, 20 bp, Artificial Sequence
  Primer b-actin
ggcccagagcaagagaggta

SEQ ID NO: 14 - DNA, 20 bp, Artificial Sequence
  Primer b-actin
ggttggccttagggttcagg

SEQ ID NO: 15 - DNA, 19 bp, Artificial Sequence
  Primer RP105
tcagtgctgccaatttccc

SEQ ID NO: 16 - DNA, 21 bp, Artificial Sequence
  Primer RP105
ctgcagcagtcagaagcctct

SEQ ID NO: 17 - DNA, 22 bp, Artificial Sequence
  Primer TLR4
agtttcctgcaatggatcaagg

SEQ ID NO: 18 - DNA, 20 bp, Artificial Sequence
  Primer TLR4
ggaccgacacaccaatgatg

SEQ ID NO: 19 - DNA, 19 bp, Artificial Sequence
  Primer Ubiquitin
cacttggtcctgcgcttga

SEQ ID NO: 20 - DNA, 24 bp, Artificial Sequence
  Primer Ubiquitin
caattgggaatgcaacaactttat

SEQ ID NO: 21 - DNA, 20 bp, Artificial Sequence
  CpG
tccatgacgttcctgatgct